(19) United States
(12) Patent Application Publication
(10) Pub. No.: US 2003/0129222 A1
(43) Pub. Date: Jul. 10, 2003

(54) Liposomal imexon

(76) Inventors: Gabriel Lopez-Berestein, Bellaire, TX (US); William A. Remers, Tucson, AZ (US); Evan Hersh, Tucson, AZ (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701, US

(21) Appl. No.: 09/989,695

(22) Filed: Nov. 20, 2001

Related U.S. Application Data
(60) Provisional application No. 60/386,215, filed on Nov. 21, 2000.

(51) Int. Cl. A61K 9/127

(52) U.S. Cl. 424/450

(57) ABSTRACT

Disclosed are novel compositions comprising a lipid and imexon or a derivative thereof. Also disclosed are liposomal compositions comprising imexon or a derivative thereof. Methods for administrating pharmaceutically acceptable compositions comprising a lipid and imexon or a derivative thereof for the treatment of diseases, such as cancer, are also disclosed herein.

The present application claims priority to U.S. Ser. No.09/721,040 filed on Nov. 21, 2000, which was converted by petition to a provisional application. The entire text of the above-referenced application is specifically incorporated herein by reference without disclaimer.

### BACKGROUND OF THE INVENTION

1. Field of the Invention

The present invention relates generally to the fields of pharmaceutical compositions and delivery. More particularly, it concerns formulation and delivery of imexon and/or hydrophobic derivatives thereof in combination with a lipid. The present invention also concerns methods of treating diseases such as cancer with imexon and/or a derivative in combination with a lipid.

2. Description of Related Art

Diseases such as cancer are a major cause of death of individuals worldwide. Hundreds of aziridines have been synthesized and screened as potential antineoplastic agents (Dermer and Ham, 1969; Goodridge et al., 1963). Most of these compounds are substituted on the aziridine nitrogen with groups such as alkyl, aralkyl, aryl, heterocyclic, acyl, alkanoic, carbamate, and dithiocarbamic acid (Denner and Ham, 1969). Clinically significant agents have two or more aziridine rings linked through nitrogen to a phosphorus (thio-TEPA) or a quinone ring (diazaquone) (Reynolds, 1995; Dorr and Von Hoff, 1994). The aziridine ring also occurs in complex natural products, such as mitomycin C, in which the aziridine nitrogen is unsubstituted (Remers and Iyengar, 1995). Generally, aziridine antitumor agents act by alkylating nucleophilic atoms on guanine residues in DNA (Reynolds, 1995).

In 1975, Bicker reported a type of carcinostatic aziridine, 2-cyanoaziridine-1-carboxamide (Formula 1) (Bicker, 1984; Bicker, 1975). This compound was active against PIE 2-3 sarcoma in Wistar rats, and it had low toxicity. Especially interesting was the observation that it increased the number of leukocytes, rather than decreasing them (Bicker, 1984). In contrast to known aziridines, it showed no alkylating activity toward 4-(4-nitrobenzyl)pyridine (Bicker, 1975). The N-phenyl derivative 13 (Table 1) was then synthesized and found active in the PIE 2-3 sarcoma model over a 10-fold dose range (Bicker and Fuchse, 1975). It is thought that that derivatives 1 and 13 (Table 1) may act indirectly by an effect on immunological mechanisms. Consequently, some cyanoaziridines including Formula 1, its N-methyl derivative 6 (Table 1), and its N-(4-sulfamylphenyl) derivative 22 (Table 1) was made and evaluated them for oral immunostimulatory activity as measured by increase in leukocyte counts in rats (Schaumann et al., 1979).

When 2-cyanoazizidine-1-carboxamide was treated with KOH in methanol, it underwent cyclization to 4-imino-3-diazabicyclo[3.1.0]hexan-2-one (2), which is named imexon (U.S. Pat. No. 4,083,987). Imexon solutions in water slowly revert partially to Formula 1. Thus, the chemistry and biological activity of imexon is closely related to that of cyanoaziridines. Various derivitives of imexon have also been described (WO 99/00120). Imexon possesses immunomodulatory properties, though it is not myelosuppressive (WO 99/00120). Imexon was active against a variety of transplanted syngeneic tumors in rodents (Bicker and Hebold, 1977). It also was active against human lymphoma, melanoma, and prostate cancer cell lines in SCID mice (Hersh et al., 1995; Salmon and Hersh, 1994; Bicker and Fuhse, 1975). Phase I clinical trials conducted in Europe in 1985 established that imexon was well-tolerated and produced minimal nausea and vomiting even in the absence of prophylactic antiemetics (Micksche et al., 1978). In one of these trials, objective responses or stabilization of the disease was found in patients with lung cancer, breast cancer, or liver cancer (Sagaster et al., 1995). It was reported that in fresh human tumor cells in clonogenic: assay, imexon was selectively cytotoxic to multiple myeloma (Hersh et al., 1992).

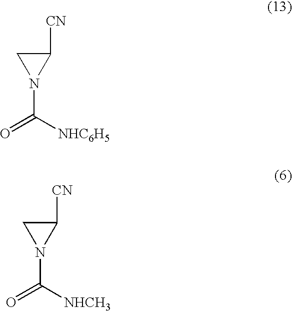

Many other derivatives of 2-cyanoaziridine have been prepared and screened for antitumor activity. Among them, azimexon (3) and ciamexon (4) received initial clinical study, but they were not successful. Azimexon is a prodrug that decomposes very quickly in water to acetone, 2-cyanoaziridine, and aziridine-2-carboxamide (Bicker, 1984). The German patent literature contains claims to numerous N-substituted 2-cyanoaziridines. These substituents include esters of carboxylic acids, acylcarboxamides, sulfonylcarboxamides, and phosphorylcarboxamides (German Patent Nos. 2,644,820, 2,727,550, 2,740,248 and 2,656,323). Some of the compounds have alkyl or phenyl substituents at position 3, and others have the cyano group replaced by carboxamide or carboxylic acid ester. No data was given on the antitumor and immunomodulatory activity.

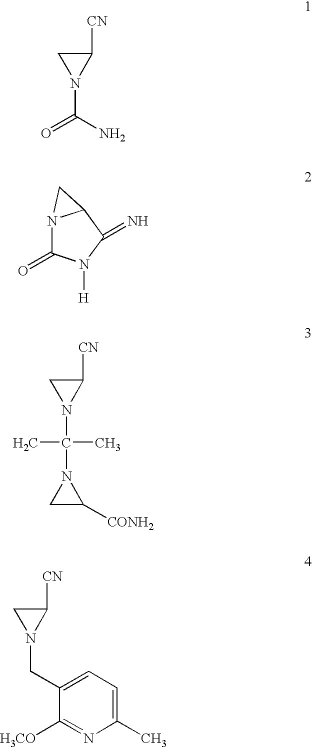

Various imexon derivatives with anti-cancer cytotoxic properties have not been fully developed due to their hydrophobicity. Given the continuing problem with cancer related deaths and the potential value of these compounds in cancer therapy, there exists a need for improved formulations and delivery of imexon related anti-cancer compositions.

### SUMMARY OF THE INVENTION

The present invention overcomes the deficiencies in the past compositions and methods by providing compositions comprising imexon and/or derivatives thereof, in combination with a lipid for to improve its therapeutic index, biodistribution and/or solubility in aqueous mediums. The present invention also overcomes past deficiencies by providing methods for preparing and administering such compositions. In particular, methods of treating such diseases as cancer with the compositions and methods are described herein.

The invention first provides a pharmaceutical composition, comprising an imexon or a derivative thereof in combination with one or more lipids.

The invention provides a pharmaceutical liposome composition, comprising an imexon or a derivative thereof in combination with one or more lipids.

The invention further provides a method of treating an individual with cancer, comprising administering to the individual a therapeutically effective amount of a composition comprising an imexon or a derivative thereof in combination with one or more lipids.

The invention also provides a method of stimulating the immune system of an individual, comprising administering a therapeutically effective amount of a composition comprising an imexon or a derivative thereof in combination with one or more lipids.

In certain embodiments, the lipid or lipids comprise at least one phospholipid. In specific aspects, the phopholipid is dimyristoyl phosphatidyl choline, dimyristoylphosphatidylglycerol, phosphatidic acid or any combination thereof. Thus, in particular aspects, the composition comprises a plurality of phospholipids. In one facet, the plurality phospholipids comprise dimyristoyl phosphatidyl choline and dimyristoylphosphatidylglycerol. In another facet, the composition comprises dimyristoyl phosphatidyl choline and dimyristoylphosphatidylglycerol in a 7:3 molar ratio. In another aspect, the plurality of lipids comprise dimyristoyl phosphatidyl choline and phosphatidic acid. In a specific facet, the composition comprises dimyristoyl phosphatidyl choline and phosphatidic acid in a 7:1 molar ratio.

In certain embodiments, at least a portion of the lipids comprise micelles. In other embodiments, at least a portion of the lipids comprise liposomes.

In some embodiments, the imexon or a derivative thereof is hydrophobic. In specific aspects, the composition comprises imexon. In other aspects, the composition comprises at least one derivative of imexon. In certain facets, the derivative of imexon comprises AMP-404. In other facets, the derivative of imexon comprises AMP-404. In some facets, the derivative of imexon comprises AMP-408. In particular facets, the derivative of imexon comprises AMP-415. In specific facets, the derivative of imexon comprises AMP-416. In additional facets, the derivative of imexon comprises AMP-419. In further facets, the derivative of imexon comprises AMP420. In yet other facets, the derivative of imexon comprises AMP-421. In other facets, the derivative of imexon comprises AMP-423. In some facets, the derivative of imexon comprises AMP-425. In certain facets, the derivative of imexon comprises AMP-403, AMP-405, AMP-406, AMP-407, AMP-408, AMP-409, AMP-410, AMP-412, AMP-413, AMP-414, AMP-417, AMP-418, AMP-419, AMP-420, AMP-421, AMP-422, AMP-423, AMP-424, AMP-425 or a combination thereof.

In some embodiments, the composition further comprises a targeting agent, a diagnostic agent, a second therapeutic agent or a combination thereof. In certain aspects, the targeting agent, diagnostic agent or second therapeutic agent is covalently attached to the lipids by a linking moeity. In specific aspects, the compositon comprises a second therapeutic agent. In some facets, the second therapeutic agent comprises an anticancer agent. In other facets, the anticancer agent is chemotherapy agent, a radiotherapy agent, an immune therapy agent, a genetic therapy agent, a hormonal therapy agent, a biological agent or a combination thereof.

In yet another embodiment, the invention relates to composition of a product comprising a lipid and an imexon and/or a derivative thereof, and further comprises at least one additional agent.

In yet another embodiment, the invention relates to composition of a product comprising a liposome and an imexon and/or a derivative thereof; and further comprises at least one additional agent

In another embodiment, the invention relates to composition of a product comprising a lipid and an imexon and/or a derivative thereof for use as a medicament and further comprises at least one additional agent for use as a medicament.

In still another embodiment, the invention relates to composition of a product comprising a liposome and an imexon and/or a derivative thereof for use as a medicament and further comprises at least one additional agent for use as a medicament.

The invention employs the use of a compound or composition comprising a lipid and an imexon and/or a derivative thereof for the manufacture or a medicament for the treatment of disease such as cancer; and further employs at least one additional agent for the manufacture or a medicament for the treatment of disease such as cancer.

In addition, the invention employs the use of a compound or composition comprising a liposome and an imexon and/or a derivative thereof for the manufacture or a medicament for the treatment of disease such as cancer; and further employs at least one additional agent for the manufacture or a medicament for the treatment of disease such as cancer.

As used herein the specification, “a” or “an” may mean one or more. As used herein in the claim(s), when used in conjunction with the word “comprising”, the words “a” or “an” may mean one or more than one, As used herein “another” may mean at least a second or more. As used herein, “any range derivable therein” means a range selected from the numbers described in the specification.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A, 1B and 1C. Cytotoxic Effect of AMP-404 on day 1, day 2 and day 3, respectively.

FIGS. 2A, 2B and 2C. Cytotoxic Effect of AMP-415 on day 1, day 2 and day 3, respectively.

FIG. 3. Correlation between substituent lipophilicity (π) and toxicity to multiple myeloma cells for 2-cyanoaziridine-1-carboxamides.

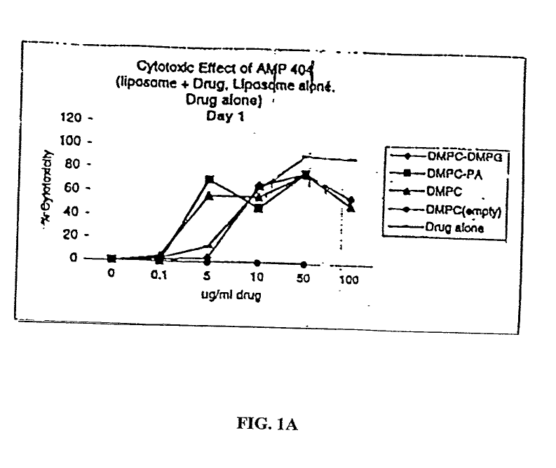

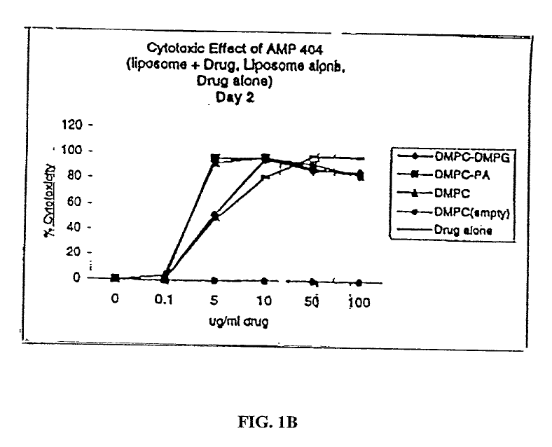

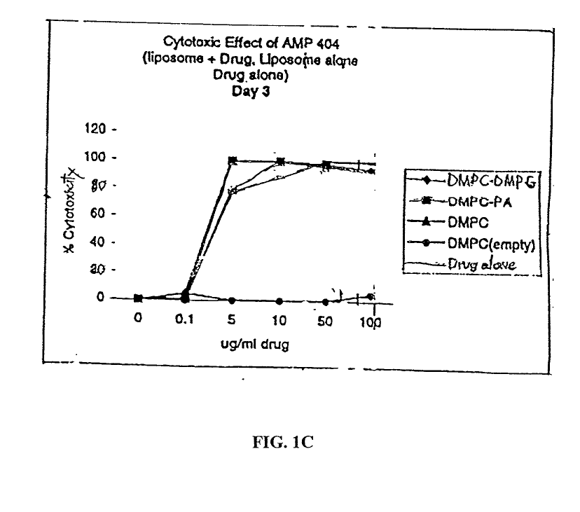

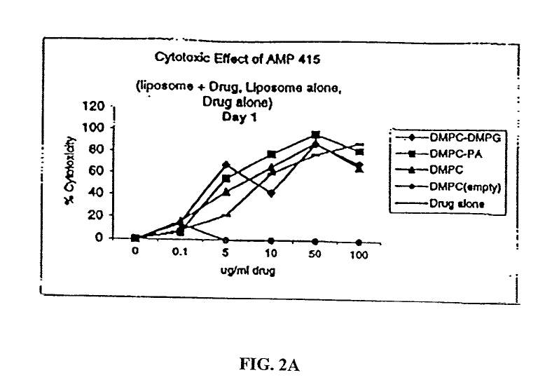

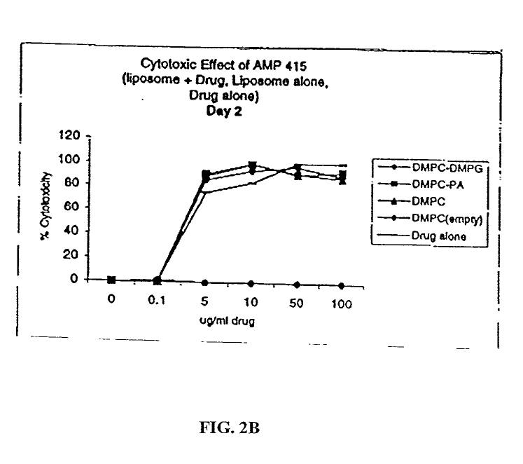

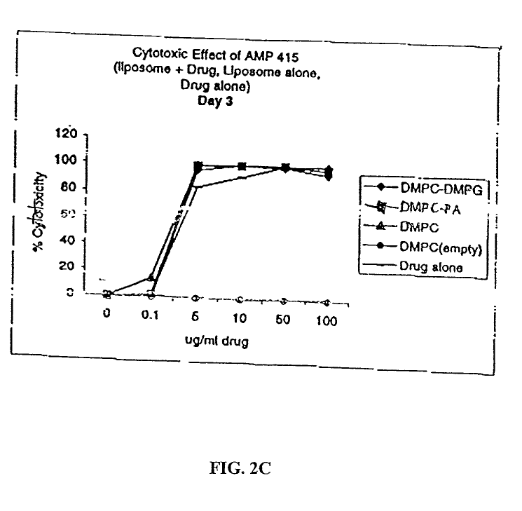

### DESCRIPTION OF ILLUSTRATIVE EMBODIMENTS

Imexon and derivatives thereof have been shown to have a broad spectrum of antitumor activities against a wide range of solid and hemtologic malignancies in vitro. Some hydrophobic derivatives of imexon seem to display better in vitro potency than imexon. However, because of their hydrophobicity, these various compounds have not been developed.

The present invention overcomes the limitations of current imexon compositions by providing lipid-imexon formulations, particularly liposome incorporated imexon and derivatives thereof, for the treatment of cancer. The imexon and imexon derivative-lipid compositions of the present invention surprisingly showed an improved activity in some instances. It is contemplated that imexon and imexon derivative/lipid compositions may possess superior pharmaceutical properties, such as for example, an improved therapeutic index or biodistribution, than other imexon formulations.

For example, it is contemplated that the biodistribution of lipid compositions of the present invention, particularly liposomes, may be altered by changing the size or lipid composition. In certain embodiments, it is contemplated that biodistribution may be altered by the conjugation of targeting ligands to the surface of the lipid composition surface, described herein. Such ligands may allow cell, tissue and/or organ specific accumulation of the drug.

In other embodiments, it is contemplated that improved toxicity profiles and/or therapeutic efficacy of imexon and/or derivatives thereof may be produced by combination with a lipid. Such aspects can be determined by procedures in cell cultures or experimental animals known to those of ordinary skill in the art or described herein. For example, one measure of drug effectiveness, cell toxicity, and safety of a compound is its therapeutic index: LD₅₀/ED₅₀. LD₅₀ is the median lethal dose, i.e., the dose lethal to 50% of the population, and ED₅₀ is the median effective dose, i.e., the dose required to achieve a specific effect in 50% of the population (e.g., antitumor activity). As would be understood by one of ordinary skill in the art, compositions having the highest therapeutic index (LD₅₀/ED₅₀) are desirable in clinical settings. In certain aspects, the therapeutic index should be greater than 2, preferably at least 10, more preferably at least 50. It is also contemplated that other pharmacokinetic or pharmacodynamic parameters (e.g., clearance, volume of distribution, half-life, drug release profiles) of the imexon and/or derivatives thereof-lipid compositions of present invention may be superior to other preparations of imexon and/or a derivative thereof. Such parameters are well known in the art (see, for example, Goodman and Gilman's “The Pharmacological Basis of Therapeutics”, pp. 18-32, 43-61, 66-78, Eighth Edition, 1990, incorporated herein by reference in relevant part).

A. Imexon

The present invention concerns various compositions comprising imexon (4-imino-1,3-diazabicyclo[3.1.0]hexan-2-one) and/or at least one derviative thereof and at least one lipid in the treatment of cancer. The compound is thereby described as being a cancerostatically-active therapeutic which displays immune-stimulating properties, and it is contemplated that the compositions of the present invention will possess like or improved activities.

As used herein, “imexon analogs” includes all known derivatives of imexon. Imexon is formed by catalytic treatment of KOH of 2-cyanoaziridine-1-carboxamide in methanol (described in Bicker, 1978; Iyengar et al., 1999, and U.S. Pat. No. 4,083,987, incorporated herein by reference). Various derivatives of imexon have been described in WO 99/00120, incorporated herein by reference. Relevant text of WO 99/00120 is shown herein below, with modifications:

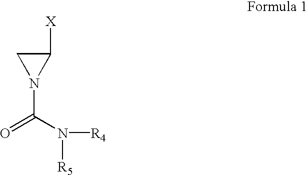

wherein

X is CN, CO₂R₁, or CONR₂R₃:

R₁ is lower alkyl, cycloalkyl, alkenyl, or aryl lower alkyl;

R₂ is hydrogen or lower alkyl;

R₃ is hydrogen lower alkyl, lower cycloalkyl, alkenyl, alkynyl, aryl, or heterocyclic ring;

R₂, R₃ and N taken together form a heterocyclic ring

R₄ is hydrogen or lower alkyl; and

R₅ is lower alkyl, lower cycloalkyl, alkenyl, alkynyl, aryl, monosubstituted aryl, disubstituted aryl, aryl lower alkyl, lower alkoxycarbonyl lower alkyl, or heterocyclic ring, with the proviso that when X is CN, and R₄ is hydrogen, then R₅ is not CH₃, C₆H₅, or, p-nitrophenyl.

R_{4,} R₅ and N taken together form a heterocyclic ring.

In particular, the invention is also directed to compound of the formula 1:

wherein X is CN, CO₂R₁ or CONR₂R₃

where R₁ is an alkyl of 1-6 carbons, a cycloalkyl of 4-7 carbons, alkenyl of 3-6 carbons or a lower alkyl substituted aryl of 7-12 carbons;

R₂ is hydrogen or lower alkyl of 1-4 carbons, and

R₃ is lower alkyl of 1-4 carbons, lower cycloalkyl of 4-7 carbons, alkenyl of 3-6 carbons, an aryl of 4-10 carbons, a substituted aryl of 4-12 carbons or heterocyclic ring of 4-16 ring members;

wherein R₄ is hydrogen or lower alkyl of 1-4 carbons; and,

wherein R₅ is an alkyl of 1-8 carbons, lower cycloalkyl of 4-7 carbons, alkenyl of 3-6 carbons, an aryl of 4-10 carbons, a substituted aryl of 4-12 carbons, a heterocyclic group of 4-16 members and where R₄, R₅ and N taken together form a heterocyclic ring of between 4 and 16 members.

The invention further includes compound of the formula 1:

wherein X is CN, CO₂R₁ or CONR₂R₃

wherein R₁ is an alkyl of 1-6 carbons, a cycloaklyl of 4-7 carbons, alkenyl of 3-6 carbons or an lower alkyl substituted aryl of 7-12 carbons;

where R₂ is hydrogen or lower alkyl of 1-4 carbons, and

where R₃ is a lower alkyl of 1-4 carbons, a lower cycloalkyl of 4-7 carbons, an alkenyl, an aryl of 4-10 carbons, a heterocyclic ring of 4-16 members or a substituted aryl or substituted heterocyclic ring where said substitutents are 1 or 2 and independently selected from the group consisting of lower alkyl of 1-4 carbons, nitro, halo substituted lower alkyls of 1-4 carbons, a lower alkyl substituted acyloxy of 1-5 carbons, a lower alkyl substituted acyl of 1-5 carbons;

wherein R₄ is hydrogen or lower alkyl of 1-4 carbons; and

wherein R₅ is an alkyl of 1-8 carbons, lower cycloalkyl of 4-7 carbons, alkenyl of 3-6 carbons, an aryl of 4-10 carbons, a substituted aryl of 4-12 carbons having 1-2 substitutents wherein the substituents are independently selected from the group consisting of lower alkyl of 1-4 carbons, nitro, halo substituted lower alkyls of 1-4 carbons, a lower alkyl substituted acyloxy of 1-5 carbons, a lower alkyl substituted acyl of 1-5 carbons, a hetrocyclic group of 4-16 members. Substitutents R₄ and R₅ may join to form a heterocyclic ring of 4-16 members.

Preferred compounds include those wherein X is CN. Additional preferred compounds are those where X is CN and R₄ is hydrogen; and R₅ is a straight chain alkyl of 1 to 8 carbons, an unsubstituted aryl, a monosubstituted or disubstituted aryl wherein the aryl is independently substituted with halo, lower alkyl, halo substituted lower alkyl, lower alkyl-substituted acyloxy or lower alkyl-substituted acyloxy.

Also preferred are those compounds wherein X is CN and R₄ is hydrogen; and R₅ is a heterocyclic group or an unsubstituted aryl. Particularly preferred are those compounds where X is CN and R₄ is hydrogen; and R₅ is a pryidyl, a phenyl, asubstituted phenyl or a naphthyl.

As used herein, the term “alkyl” when used alone or in combination, consists of a carbon chain containing from one to eight carbon atoms. The alkyl groups may be a straight chain or a branched chain. It includes such groups as methyl, ethyl, propyl, isopropyl, n-butyl, sec-butyl, isobutyl, t-butyl, n-pentyl, amyl, n-hexyl, and the like. The preferred alkyl groups are methyl and ethyl. Lower alkyls are C1-4 and higher alkyls are C₅-C₈. “Substituted alkyl” refers to alkyl as just described including one or more functional groups such as lower alkyl, aryl, acyl, halogen (i.e., alkylhalos, e.g., CF₃), hydroxy, amino, alkoxy, alkylamino, acylamino, acyloxy, aryloxy, aryloxyalkyl, mercapto, both saturated and unsaturated cyclic hydrocarbons, heterocycles and the like. These groups may be attached to any carbon of the alkyl moiety.

The term “aryl” is used herein to refer to an aromatic substituent which may be a single aromatic ring or multiple aromatic rings which are fused together, linked covalently, or linked to a common group such as a methylene or ethylene moiety. The common linking group may also be a carbonyl as in benzophenone. The aromatic ring(s) may include phenyl, napthyl, biphenyl, diphenylmethyl and benzophenone among others. The term “aryl” encompasses “arylalkyl.”

The term “arylalkyl” is used herein to refer to a subset of “aryl” in which the aryl group is attached to the nucleus shown in Formula 1 by an alkyl group as defined herein.

“Substituted aryl” refers to aryl as just described including one or more functional groups such as lower alkyl, acyl, halogen, alkylhalos (e.g., CF₃), hydroxy, amino, alkoxy, alkylamino, acylamino, acyloxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The term “substituted aryl” encompasses “substituted arylalkyl.”

“Substituted arylalky” defines a subset of “substituted aryl” wherein the substituted aryl group is attached to the nucleus shown in Formula 1 by an alkyl group as defined herein.

The term “acyl” is used to describe a ketone substituent, —C(O)R, where R is alkyl or substituted alkyl, aryl or substituted aryl as defined herein.

The term “halogen” is used herein to refer to fluorine, bromine, chlorine and iodine atoms.

The term “hydroxy” is used herein to refer to the group —OH.

The term “amino” is used to describe primary amines, R—NH₂.

The term “alkoxy” is used herein to refer to the —OR group, where R is a lower alkyl, substituted lower alkyl, aryl, substituted aryl, arylalkyl or substituted arylalkyl wherein the alkyl, aryl, substituted aryl, arylalkyl and substituted arylalkyl groups are as described herein. Suitable alkoxy radicals include, for example, methoxy, ethoxy, phenoxy, substituted phenoxy, benzyloxy, phenethyloxy, t-butoxy, etc.

The term “alkylamino” denotes secondary and tertiary amines wherein the alkyl groups may be either the same or different and may consist of straight or branched, saturated or unsaturated hydrocarbons.

As used herein, the term “acylamino” describes substituents of the general formula RC(O)NR′, wherein R′ is a lower alkyl group and R represents the nucleus shown in Formula 1 or an alkyl group, as defined herein, attached to the nucleus.

The term “acyloxy” is used herein to describe an organic radical derived from an organic acid by the removal of the acidic hydrogen. Simple acyloxy groups include, for example, acetoxy, and higher homologues derived from carboxylic acids such as ethanoic, propanoic, butanoic, etc. The acyloxy moiety may be oriented as either a forward or reverse ester (ie., RC(O)OR′ or R′C(O)R, respectively, wherein R comprises the portion of the ester attached either directly or through an intermediate hydrocarbon chain.

As used herein, the term “aryloxy” denotes aromatic groups which are linked to the nucleus shown in Formula 1 directly through an oxygen atom. This term encompasses “substituted aryloxy” moieties in which the aromatic group is substituted as described above for “substituted aryl.”

As used herein “aryloxyalkyl” defines aromatic groups attached, through an oxygen atom to an alkyl group, as defined herein. The alkyl group is attached to the nucleus shown in Formula 1. The term “aryloxyalkyl” encompasses “substituted aryloxyalkyl” moieties in which the aromatic group is substituted as described for “substituted aryl.”

As used herein, the term “mercapto” defines moieties of the general structure R-S-R′ wherein R and R′ are the same or different and are alkyl, aryl or heterocyclic as described herein.

The term “saturated cyclic hydrocarbon” denotes groups such as the cyclopropyl, cyclobutyl, cyclopentyl, etc., and substituted analogues of these structures.

The term “unsaturated cyclic hydrocarbon” is used to describe a monovalent non-aromatic group with at least one double bond, such as cyclopentene, cyclohexene, etc. and substituted analogues thereof.

The term “heteroaryl” as used herein refers to aromatic rings in which one or more carbon atoms of the aromatic ring(s) are substituted by a heteroatom such as nitrogen, oxygen, or sulfur. Heteroaryl refers to structures which may be a single aromatic ring, multiple aromatic ring(s), or one or more aromatic rings coupled to one or more non-aromatic ring(s). In structures having multiple rings, the rings can be fused together, linked covalently, or linked to a common group such as a methylene or ethylene moiety. The common linking group may also be a carbonyl as in phenyl pyridyl ketone. As used herein, rings such as thiophene, pyridine, isoxazole, phthalimide, pyrazole, indole, furan, etc, or benzo-flised analogues of these rings are defined by the term “heteroaryl.”

“Heteroarylalkyl” defines a subset of “heteroaryl” wherein an alkyl group, as defined herein, links the heteroaryl group to the nucleus shown in Formula 1.

“Substituted heteroaryl” refers to heteroaryl as just described wherein the heteroaryl nucleus is substituted with one or more functional groups such as lower alkyl, acyl, halogen, alkylhalos (e.g., CF₃), hydroxy, amino, alkoxy, alkylamino, acylamino, acyloxy, mercapto, etc. Thus, substituted analogues of heteroaromatic rings such as thiophene, pyridine, isoxazole, phthalimide, pyrazole, indole, furan, etc. or benzo-fused analogues of these gins are defined by the term “substituted heteroaryl.”

“Substituted heteroarylalkyl” refers to a subset of “substituted heteroaryl” as described above in which an alkyl group, as defined herein, links the heteroaryl group to the nucelus as shown in Formula 1.

The term “heterocyclic” is used herein to describe a monovalent saturated or unsaturated non-aromatic group having a single ring or multiple condensed rings from 1-12 carbon atoms and from 1-4 heteroatoms selected from nitrogen, sulfur or oxygen within the ring. Such heterocycles are, for example, tetrahydrofuran, morpholine, piperidine, pyrrolidine, etc.

The term “substituted heterocyclic” as used herein describes a subset of “heterocyclic” wherein the heteroycle nucleus is substituted with one or more functional groups such as lower alkyl, acyl, halogen, alkylhalos (e.g., CF₃), hydroxy, amino, alkoxy, alkylamino, acylamino, acyloxy, mercapto, etc. It is preferred that the heterocyclic ring contain 5 or 6 ring atoms.

The term “heterocyclicalkyl” defines a subset of “heterocyclic” wherein an alkyl group, as defined herein, links the heterocyclic group to the nucleus shown in Formula 1.

The term “substituted heterocyclicalkyl” defines a subset of “heterocyclic alkyl” wherein the heterocyclic nucleus is substituted with one or more functional groups such as lower alkyl, acyl, halogen, alkylhalos (e.g., CF₃), hydroxy, amino, alkoxy, alkylamino, acylamino, acyloxy, mercapto, etc.

“Alkylene” refers herein to a divalent lower alkyl substituent as defined above, such as methylene (—CH₂—), ethylene (—CH₂CH₂—) or propylene (—CH₂CH₂CH₂—). “Substituted alkylene” refers to alkylene as just described including one or more functional groups such as lower alkyl, aryl, aralkyl, acyl, halogen, hydroxyl, amino, acylamino, acyloxy, alkoxyl, mercapto and the like.

“Alkenylene” refers herein to a divalent lower alkyl substituent having one or more double bonds, such as ethenylene (—CH═CH—). “Alkynylene” refers herein to a divalent lower alkyl substituent having one or more triple bonds, such as ethynylene (—C═C—). “Substituted alkenylene” and “substituted alkynylene” refer to an alkenylene or an alkynylene as just described including one or more functional groups such as lower alkyl, aryl, aralkyl, acyl, halogen, hydroxyl, amino, acylamino, acyloxyl, alkoxyl, mercapto and the like.

Unless otherwise stated: (i) all numerical ranges are inclusive, ie., 1-3 or 1 to 3 carbons includes 1, 2 and 3 carbons; (ii) heterocyclical substituents may be attached through any available hydrogen that would exist in the non-radical form of the heterocycle member.

The basic nucleus of Formula II, wherein X is CN can be made in accordance with the method of Jänisch el al., (Jänisch el al., 1992). Briefly, 2,3-dibromopropionitrile is treated at 5-15° C. with ammonia and then triethanolamine is added and the mixture is heated at reflux temperature. Following workup, the product is distilled under reduced pressure.

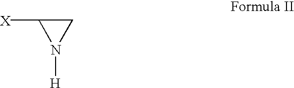

When X is CO2R1, the basic nucleus is made by the method of Kyburz et al., (Kyburz et al., 1968). In this method, esters of 2,3-dibromopropionic acid are stirred with N-phenyl-2-naphthylamine. The ammonia is then evaporated and the product is worked up and distilled under reduced pressure. A variant of this method uses the corresponding esters of 2-bromoacrylic acid in place of the esters of 2,3-dibromopropionic acid. (Kyburz et al., 1968).

Compounds containing the basic nucleus wherein X is CONR2R3 are made by treating methyl or ethyl aziridine-2-carboxylate, prepared as described above, with ammonia or appropriate amines in methanol solution. In the case where R2 and R3 are H, evaporation of solvent gave the product quantitatively according to (Kyburz et al., 1968). If purification is necessary, the products may be distilled at low pressure (1-10 mm), or recrystallized. Many appropriate amines are commercially available. They include, but are not limited to alkyl (methylamine, etc.), dialkyl (diethylamine, etc.), alkenyl (allylamine), alkynyl (propargylamine), aryl (aniline, etc.), and heterocyclic (pyrrolidine, etc.)

Compounds possessing the basic nucleus can be converted into the compounds of this invention by two different methods. The preferred method depends on the structure of the product and the availability of appropriate isocyanate reagents.

When R4 is H and isocyanates are commercially available or easily prepared, the preferred method is treatment of the basic nucleus with an isocyanate in an inert solvent such as benzene or toluene until complete disappearance of the starting material is indicated by thin-layer chromatography. Generally the product crystallizes when the reaction mixture is cooled. If not, the solvent is removed under reduced pressure to provide the product. Many appropriate isocyanates are commercially available. They include, but are not limited to alkyl (methylisocyanate, etc.), lower cycloalkyl (cyclohexylisocyanate), alkenyl (allyisocyanate), aryl (phenylisocyanate), monosubstituted aryl (tolyisocyanate, etc.), disubstituted aryl (3, 4-dichlorophenylisocyanate), aryl lower alkyl (benzylisocyanate), and lower alkoxycarbonyl lower alkyl (CH2CO2C2H5). In other cases, the isocyanate can be prepared from an available intermediate. For example, 3-pyridylisocyanate is made by heating nicotinic acid azide in toluene by the procedure of Hyden and Wilbert, 1967).

When R4 is not H or when an appropriate isocyanate is not available, the preferred method for preparing the compounds of this invention is to first convert the basic nucleus into a carbamate by treating it with 1 to 1.2 equivalents of a chloroformate such as 4-nitrophenyl chloroformate or trichloromethyl chlorofonnate (reaction 1) in an inert solvent such as benzene, chloroform, or tetrahydryofuran at low temperature (5-20° C.) and removing the solvent under reduced presssure.

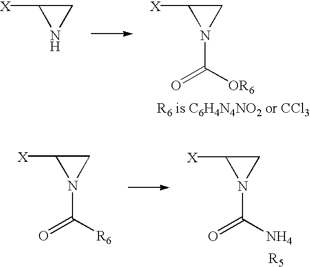

The resulting carbamate is treated with appropriate primary or secondary amines to give the desired product (reaction 2). Conditions for conducting this reaction are to treat the carbamate with 1 to 1.2 equivalents of the amine in an inert solvent such as toluene, chloroform, or tetrahydrofuran at room temperature until thin-layer chromatography indicates complete consumption of the carbamate. Many appropriate amines are commercially available. They include, but are not limited to dialkyl (diethylamine, etc.), alkynyl (propargylamine), monosubstituted aryl (2-napthylamine), disubstituted aryl (4-aminobenzoic), aryl lower alkyl (phenethylamine), heterocyclic (piperidine, etc.), and heteroaryl (2-aminothiazole).

The compounds of this invention can be purified by recrystallization from appropriate chemically inert solvents such as toluene, chloroform, and ethyl acetate.

Table 1A shows the designation numbers of imexon analogs (AMPs), their numbers used in subsequent tables and illustrations of their respective chemical formulae. When an AMP designation number is used herein, its chemical formula can be found in Table 1A.

TABLE 1A
AMP Designation of Imexon Analogs and
Their Respective Chemical Formulae:

| Compound | R |
|---|---|
| AMP-400 (2) | (imexon) |
| 403 (6) | CH₃ |
| 404 (13) | C₆H₅ |
| 405 | COCl₃ |
| 406 (7) | C₂H₅ |
| 407 (17) | 2,4-C₆H₃Cl₂ |
| 408 (18) | 3,4-C₆H₃Cl₂ |
| 409 (1) | H (intermed.) |
| 410 (9) | C(CH₃)₃ |
| 412 (11) | CH₂C₆H₅ |
| 413 (10) | C-C₆H₁₁ |
| 414 (8) | C₄H₉ |
| 415 (14) | 4-FC₆H₄ |
| 416 (15) | 4-CF₃C₆H₄ |
| 417 (16) | P-O₂NC₆H₄ |
| 418 | Bis-cyanoaziridine |
| 419 (18) | p-C₂H₅OCOC₆H₄ |
| 420 (12) | C₂H₅OCOCH₂ |
| 421 (24) | C₅H₄N (pyr.) |
| 422 (22) | H₂NSO₂C₆H₄ |
| 423 (23) | L-Naphthyl |
| 424 (21) | 2-CH₃CO₂C₆H4 |
| 425 (20) | 3-CH₃COC₆H₅ |

In addition to the compounds described above, the present invention also contemplate embodiments wherein the derivative of imexon comprises the following formula:

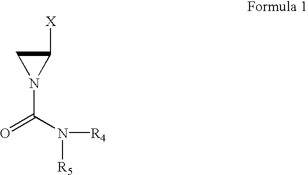

wherein

X is CN, CO₂R₁, or CONR₂R₃:

R₁ is lower alkyl, cycloalkyl, alkenyl, or aryl lower alkyl;

R₂ is hydrogen or lower alkyl;

R₃ is hydrogen lower alkyl, lower cycloalkyl, alkenyl, alkynyl, aryl, or heterocyclic ring;

R₂, R₃ and N taken together form a heterocyclic ring

R₄ is hydrogen or lower alkyl; and

R₅ is lower alkyl, lower cycloalkyl, alkenyl, alkynyl, aryl, monosubstituted aryl, disubstituted aryl, aryl lower alkyl, lower alkoxycarbonyl lower alkyl, or heterocyclic ring, with the proviso that when X is CN, and R₄ is hydrogen, then R₅ is not CH₃, C₆H₅, or, p-sulfamyl.

In certain embodiments, AMP-15 (comprising p-fluorophenyl) is a particularly preferred imexon derivative for the compositions and methods of the present invention.

In other embodiments, the present invention concerns various compositions comprising one or more 2-cyanoaziridine-1-carboxamide derivatives of imexon and at least one lipid in the treatment of cancer. Various 2-cyanoaziridine-1-carboxamide derivatives of imexon and methods of making such have been described in Iyengar et al., 1999, incorporated herein by reference.

1. General Method of Preparation of 2-Cyanoaziridine-1-carboxamides

2-Cyanoaziridine (5, Scheme 1) was prepared according to the published procedure, which involves treating ethyl 2,3-dibromopropionitrile with ammonia in methanol followed by adding triethanolamine and heating (Jähnisch et al., 1992). Conversion of 5 into the desired N-substituted 1-carboxamide derivatives 6-21, 23, and 24 (Table 1B) was effected simply by stirring it with the appropriate isocyanate in toluene or benzene at ice-bath temperature (Scheme 1). To an ice-cooled mixture of 2-cyanoaziridine (5) and toluene was added an ice-cold solution of an isocyanate (1.05 equiv) in toluene at a rate to keep the temperature below 5° C. The mixture was stirred for 1 hr in an ice bath and then placed in a refrigerator overnight. The resulting precipitate was collected, washed with toluene, and dried under vacuum to give the product. The yields and properties of these products are given in Table 1B. For the data shown on Table 1B, melting points were recorded on a Mel-Temp melting point apparatus and are uncorrected. ¹H NMR spectra were recorded on a Bruker 250 WM spectrometer, and absorptions are reported downfield from Me₄Si (δ values in ppm). Mass spectra were recorded on a Varian-MAT311 spectrometer. Elemental analyses were performed by Desert Analytics, Inc., Tucson, Ariz. For cases 6-21 the isocyanates were commercially available (Scheme 1, shown below).

TABLE 1B
Properties of New 2-Cyanoaziridine^{a}

| | | Yield, | Solvent | | |
|---|---|---|---|---|---|
| No. | R | % | impurity | mp, ° C. | ¹H NMR signals, δ (ppm from TMS) |
| 6 | CH₃ⁱ | 94 | | 98-100 | 2.47 (d, 1, J = 3 Hz), 2.57 (d, 1, J = 6 Hz), 2.8 (d, 3, J = 5 Hz) |
| | | | | | 3.05 (dd, 1, J = 6, 3 Hz), 6.18 (br, sl, NH)b |
| 7 | C₂H₅^{j} | 63 | | 58-62 | 1.1 (t, 3, 5 Hz), 2.4 (d, 1, J = 3 Hz), 2.50 (d, 1, J = 6 Hz), 2.97 (dd, 1, |
| | | | | | J = 6 Hz, 3 Hz), 3.3 (q, 2, J = 6 Hz), 6.1 (brs, 1, NH)^{b} |
| 8 | n-C₄H₉ | 92 | 0.1H₂O | 98-102 | 1.0 (t, 3) 1.4 (m, 2), 1.6 (m, 2), 2.5 (d, 1, J = 3 Hz), 2.55 (d, 1, |
| | | | | | J = 6 Hz), 3.0 (dd, 1, J = 6, 3 Hz), 3.2 (m, 2), 6.1 (br s, 1, NH)^{b} |
| 9 | t-C₄H₉ | 81 | | 46-48 | 1.4 (s, 9), 2.84 (d, 1, J = 3 Hz), 2.86 (d, 1, J = 6 Hz), 3.0 (dd, 1, J = 6, |
| | | | | | 3 Hz), 5.8 (br s, 1, HN)^{c} |
| 10 | c-C₆H₁₁ | 64 | | 98-102 | 1.2-1.5 (m, 5), 1.6-2.1 (m, 5), 2.45 (d, 1 J = 3 Hz), 2.53 (d, 1, |
| | | | | | J = 6 Hz). 3/015 (dd, 1, J = 6, 3 Hz), 3.6 (m, 1), 5.8 (br s, 1 NH)^{b} |
| 11 | C₆H₅CH₂ | 25 | | 42-44 | 2.36 (d, 1, J = 3 Hz), 2.46 (d, 1, J = 6 Hz), 2.93 (dd, 1,J = 6, 3 Hz), |
| | | | | | 4.4 (d, 2), 6.8 (br s, 1, NH), 7.4 (m, 5)^{b} |
| 12 | C₂H₅OCOCH₂ | 50 | 0.75CH₃O | oil | 1.22 (t, 3 J = 7 Hz), 2.49 (d, 1, J = 3 Hz), 2.56 (d, 1, J = 6 Hz), 3.07 |
| | | | H^{d} | | (dd, J = 6 Hz), 3.91 (d, 2), 4.15 (q, 2, J = 7 Hz), 6.74 (t, 1, NH)^{b} |
| 13 | C₆H₅^{k} | 71 | | 88-90 | 2.65 (d, 1, J = 3 Hz), 2.69 (d, 1, J = 6 Hz), 3/57 (dd. 1. J = 6, 3 Hz), |
| | | | | | 7.05 (t, 1), 7.45 (d, 2), 7.60 (d, 2, 10.2 (br s, 1, NH)^{c} |
| 14 | 4-FC₆H₄ | 54 | | 99-100 | 2.55 (d, 1, J = 3 Hz), 2.68 (d, 1, J = 6 Hz), 3.20 (dd, 1, J = 6, 3 Hz). |
| | | | | | 7.0 (d, 2, J = 9 Hz), 7.5 (d, 2, J = 9 Hz), 10.2 (br s, 1, NH)^{c} |
| 15 | 4-CF₃C₆H₄ | 91 | | 166-168 | 2.62 (d, 1, J = 3 Hz), 2.74 (d, 1, J = 6 Hz), 3/32 (dd. 1. J = 6, 3 Hz). |
| | | | | | 7.54 (d. 2. 7 = 9 Hz), 7.74 (d, 2, J = 9 Hz), 10.2 (br s, 1, NH)^{c} |
| 16 | 4-NO₂C₆H₄ | 89 | 0.1H₂O | >230 | 2.77 (d, 1, J = 3 Hz), 2.81 (d, 1, J = 6 Hz), 3.69 (dd, 1, J = 6, 3 Hz), |
| | | | | dec | 7.8 (d, 2, J = 9 Hz), 8.2 (d, 2, J = 9 Hz), 10.8 br s, 1, NH)^{c} |
| 17 | 2,4-Cl₂C₆H₃ | 50 | | 110-114 | 2.70 (d, 1, J = 3 Hz), 2.71 (d, 1, J = 6 Hz), 3.57 (dd, 1, J = 6, 3 Hz), |
| | | | | | 7.44 (d, 1, dd, J = 3 Hz, J = 6 Hz), 7.57 (d, 1, J = 6 Hz), 7.68 (d, 3 |
| | | | | | Hz, 10.0 (br s, 1, NH)^{c} |
| 18 | 3,4-Cl₂C₆H₃^{e} | 76 | | 132-134 | 2.71 (d, 1, J = 3 Hz), 2.73 (d, 1, J = 6 Hz), 3.62 (dd, 1, J = 6, 3 Hz), |
| | | | | | 7.5 (dd, 1, J = 9, 3 Hz), 7.6 (d, 1, J = 9 Hz), 7.9 (d, 1, J = 3 Hz), 10.6 |
| | | | | | (br s, 1, NH)^{c} |
| 19 | 4-C₂H₅OCOC₆H₄ | 90 | | 162-165 | 1.3 (t, 3, J = 6 Hz), 2.72 (d, 1, J = 3 Hz), 2.76 (d, 1, J = 6 Hz), 3.64 |
| | | | | | (dd, 1, J = 6, 3 Hz), 4.3 (q, 2, J = 6 Hz). 7.69 (d, 2, J = 9 Hz), 7.73 (d, |
| | | | | | 2, J = 9 Hz), 10.63 (br s, 1, NH)^{c} |
| 20 | 3-CH₃COC₆H₅^{f} | 74 | | 110-112 | 2.6 (s, 3), 2.71 (d, 1, J = 3 Hz), 2.74 (d, 1 J = 6 Hz), 3.63 (dd, 1, |
| | | | | | J = 6, 3 Hz), 7.5 (t, 1, J = 9 Hz), 7.7 (d, 1 J = 9 Hz), 7.85 (d, 1, J = 9 |
| | | | | | Hz), 8.1 (s, 1), 10.5 (br s, NH)_{b} |
| 21 | 2-CH₃CO₂C₆H₄ | 10 | | 101-102 | 2.38 (s, 3), 2.55 (br s, 1), 2.64 (br s, 1), 3.20 (br s, 1), 7.15 (br s, 2), |
| | | | | | 7.2-7.6 (br s, 1), 7.68 (br s, 1), 7.96 (br s, 1, NH)^{c,h} |
| 22 | 4-H₂NSO₂C₆H₄^{g} | 39 | | 170-174 | 2.72 (d, 1, J = 3 Hz), 2.74 (d, 1, J = 6 Hz), 3.7 (dd, 1, J = 6, 3 Hz), |
| | | | | | 7.26 (s, 2, NH₂), 7.60 (d, 2), 7.73 (d, 2), 10.6 (br s, 1, NH)^{c} |
| 23 | 1-C₁₀H₇ | 56 | | 98-100 | 2.6 (br s, 1), 3.2 (br s, 1), ^{a}7.4 (br s, 1), 7.5 (m, 3), 7.7 (br s, 1), 7.8 (br |
| | | | | | s, 2), 8.1 (br s, NH)^{b,h} |
| 24 | 3-C₅H₄N | 10 | 0.2H₂O | 205 dec | 2.72 (d, 1, J = 3 Hz), 2.76 (d, 1, J = 6 Hz), 3.65 (dd, 1, J = 6, 3 Hz), |
| | | | | | 7.36 (dd, J = 3 Hz, 9 Hz, 1), 7.97 (dd, J = 6, 3 Hz, 1), 8.28 (dd, J = 3, |
| | | | | | 6 Hz), 8.71 (s, 1), 10.5 (br s, 1, NH)^{c} |

^{a}Analytical results were within ±0.40% of theoretical values for all elements (C, H, N, Cl, S, and F), except as shown in subsequent footnotes. In some examples, water or the solvent impurities indicated in the table had to be added to reconcile the calculated and found values for these elements.
^{b}The solvent was CDCl₃.
^{c}The solvent was DMSO-d₆.
^{d}The product was eluted from TLC plate scrapings by CH₃OH.
^{e}N: calcd, 16.40; found, 15.68.
^{f}N: calcd, 18.32; found, 17.97.
^{g}N: calcd, 21.04; found, 19.60.
^{h}The expected doublets were not resolved and appeared as broad singlets.
ⁱMS (EI) 125 (M⁺).
^{j}MS (EI) 139 (M⁺).
^{k}MS (EI) 187 (M⁺).

In certain other cases, the isocyanates had to be synthesized. Thus, 2-acetylphenyl isocyanate (27) was prepared by treating 2-acetoxybenzoyl chloride (25) with sodium azide, and the resulting acid azide (26) was heated in benzene under nitrogen at 70-75° C. 3-Pyridyl isocyanate (32) was made from nicotinic acid hydrazide (30) by way of nicotinic azide (31) according to the published method (Hyden and Wilbert, 1967). In the synthesis of N-(4-sulfamylphenyl) analogue 22, 2-cyanoaziridine (5) was condensed with 4-chlorosulfonylphenyl isocyanante 28 and the resulting intermediate 29 was treated with liquid ammonia (Scheme 1).

2-Cyanoaziridine-1-[N-[ethoxycarbonyl)methyl]carboxamide] (12) was a colorless oil that did not crystallize on cooling. It was dissolved in cold chloroform and diluted with cold hexane. The mixture was stirred briefly, then the solvent was decanted, and the oil was dried under vacuum.

2. 2-Cyanoaziridine-1-[N-(2-acetoxy-phenyl)carboxamide]

2-Acetylbenzoic acid azide (26) was prepared by treating 2-acetoxybenzoyl chloride (25) with 1.1 equiv of sodium azide in acetone and water at 0-5° C. for 24 hr. It had a peak at 2245 cm⁻¹ in the IR spectrum. Without further purification, it was heated in benzene at 70-75° C. under nitrogen for 2 hr to give 2-acetylphenyl isocyanate (27).

This crude isocyanate was converted into 21 by the general procedure, and the overall yield was 10%.

3. 2-Cyanoaziridine-1-[N-(4-sulfamyl-phenyl)carboxamide]

2-Cyanoaziridine-1-(N-(4-chlorosulfonylphenyl)carboxamide] (24) was prepared from 2-cyanoaziridine (29) and 4-chlorosulfonylphenyl isocynate (28) by the general procedure. It had mp 142-144° C. Without further purification, it was converted by treatment with excess liquid ammonia into 22 in an overall yield of 39%.

4. 2-Cyanoaziridine-1-[N-(3-pyridyl)carboxamide)

3-Pyridyl isocyanate (32) was prepared from nicotinic acid hydrazide (30) by way of nicotinic acid azide (31) according to the published procedure (German Patent No. 2,656,323). This intermediate was converted into 24 by the general method, except that the solvent was benzene.

B. Lipid Compositions

In certain embodiments, the present invention concerns a novel composition comprising one or more lipids associated with at least one imexon and/or a derivative thereof. A lipid is a substance that is characteristically insoluble in water and extractable with an organic solvent. Compounds than those specifically described herein are understood by one of skill in the art as lipids, and are encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphotidyl serine, a phosphatidyl ethanolamine, a phosphatidyl choline, a phosphatidyl glycerol or a phosphatidyl inositol. Other phospholipids include a phosphatidic acid or a diacetyl phosphate. In one aspect, a phosphatidylcholine comprises a dioleoylphosphatidylcholine (a.k.a cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g, lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

2. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

3. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

4. Lipid Composition Structures

An imexon and/or a derivative thereof associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid/imexon and/or a derivative thereof associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL)-imexon and/or derivative thereof or Superfect (Qiagen)-imexon or a derivative thereof complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

a. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

b. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al., 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

5. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or an imexon and/or a derivative thereof may be, for example, encapsulated in the aqueous interior of a liposome, interspersed within the lipid bilayer of a liposome, attached to a liposome via a linking molecule that is associated with both the liposome and the imexon and/or derivative thereof, entrapped in a liposome, complexed with a liposome, etc.

a. Making Liposomes

A liposome used according to the present invention can be made by different methods, as would be known to one of ordinary skill in the art.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the imexon and/or a derivative thereof, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the imexon and/or a derivative thereof or other agent is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983; Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000 ×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al., 1987; Mayhew et al., 1984; Cheng et al., 1987; and Liposome Technology, 1984, each incorporated herein by reference).

A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/imexon and/or a derivative thereof only of appropriate and uniform size, which are structurally stable and produce maximal activity. Such techniques are well-known to those of skill in the art (see, for example Martin, 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

b. Liposome Targeting

Association of a imexon and/or derivatives thereof with a liposome may improve biodistribution and other properties of the compositions of the present invention. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposome/imexon and/or a derivative thereof composition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of an imexon and/or a derivative thereof. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

i. Cross-linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groupg. By taking advantage of the differential reactivitieg of the two different functional groups, cross-linking can be controlled both selectively and sequentially. The bifunctional cross-linking reagents can be divided according to the specificity of their functional groups, e.g., amino, sulthydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. Nos. 5,603,872 and 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 2 details certain hetero-bifunctional cross-linkers considered useful in the present invention

TABLE 2
HETERO-BIFUNCTIONAL CROSS-LINKERS

| | | | Spacer Arm Length/ |
|---|---|---|---|
| Linker | Reactive Toward | Advantages and Applications | after cross-linking |
| SMPT | Primary amines Sulfhydryls | Greater stability | 11.2 A |
| SPDP | Primary amines Sulfhydryls | Thiolation | 6.8 A |
| | | Cleavable cross-linking | |
| LC-SPDP | Primary amines Sulfhydryls | Extended spacer arm | 15.6 A |
| Sulfo-LC-SPD | Primary amines Sulfhydryls | Extended spacer arm | 15.6 A |
| P | | Water-soluble | |
| SMCC | Primary amines Sulfhydryls | Stable maleimide reactive group | 11.6 A |
| | | Enzyme-antibody conjugation | |
| | | Hapten-carrier protein conjugation | |
| Sulfo-SMCC | Primary amines Sulfhydryls | Stable maleimide reactive group | 11.6 A |
| | | Water-soluble | |
| | | Enzyme-antibody conjugation | |
| MBS | Primary amines Sulfhydryls | Enzyme-antibody conjugation | 9.9 A |
| | | Hapten-carrier protein conjugation | |
| Sulfo-MBS | Primary amines Sulfhydryls | Water-soluble | 9.9 A |
| SIAB | Primary amines Sulfhydryls | Enzyme-antibody conjugation | 10.6 A |
| Sulfo-SIAB | Primary amines Sulfhydryls | Water-soluble | 10.6 A |
| SMPB | Primary amines Sulfhydryls | Extended spacer arm | 14.5 A |
| | | Enzyme-antibody conjugation | |
| Sulfo-SMPB | Primary amines Sulfhydryls | Extended spacer arm | 14.5 A |
| | | Water-soluble | |
| EDC/Sulfo-N | Primary amines Carboxyl | Hapten-Carrier conjugation | 0 |
| HS | groups | | |
| ABH | Carbohydrates Nonselective | Reacts with sugar groups | 11.9 A |

In instances where a particular polypeptide does not contain a residue amenable for a given cross-linking reagent in its native sequence, conservative genetic or synthetic amino acid changes in the primary sequence can be utilized.

ii. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., 1986). In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, an imexon and/or a derivative thereof composition may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific imexon and/or a derivative thereof delivery and/or targeting vehicle may comprise a specific binding ligand in combination with a lipid, particularly comprised in a liposome. The imexon and/or derivative thereof to be delivered are housed within a liposome and the specific binding ligand is functionally incorporated into a lipogome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and an imexon and/or a derivative thereof/lipid composition. Others comprise a cell receptor-specific ligand to which an imexon and/or a derivative thereof/lipid composition to be delivered has been operatively attached. For example, several ligands have been used for receptor-mediated gene transfer (Wu and Wu, 1987; Wagner et al., 1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

c. Liposome/Nucleic Acid Combinations

It is contemplated that when the liposome/imexon and/or derivatives thereof composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich etal., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E. coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

C. Cancer Tratents

In order to increase the effectiveness of an imexon and/or a derivative thereof/lipid composition, it may be desirable to combine these compositions of the with an agent effective in the treatment of hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with an imexon and/or a derivative thereof/lipid composition effective to kill or inhibit proliferation of a cancer cell. This process may involve contacting the cell(s) with an agent(s) and the imexon and/or a derivative thereof/lipid composition at the same time. This may be achieved by contacting the cell, tissue or organism with a single composition or pharmacological formulation that includes both an imexon and/or a derivative thereof/lipid composition and one or more agents, or by contacting the cell with two or more distinct compositions or formulations, at the same time, wherein one composition includes an imexon and/or a derivative thereof/lipid composition and the other includes one or more agents.

The terms “contacted” and “exposed,” when applied to a cell, tissue or organism, are used herein to describe the process by which a therapeutic construct of the present invention and/or another agent, such as for example a chemotherapeutic or radiotherapeutic agent, are delivered to a target cell, tissue or organism or are placed in direct juxtaposition with the target cell, tissue or organism. To achieve cell killing or stasis, the imexon and/or derivative thereof/lipid composition and/or additional agent(s) are delivered to one or more cells in a combined amount effective to kill the cell(s) or prevent them from dividing.

The imexon and/or derivative thereof/lipid composition may precede, be co-current with and/or follow the other agent(s) by intervals ranging from minutes to weeks. In embodiments where the imexon and/or derivative thereof/lipid composition, and other agent(s) are applied separately to a cell, tissue or organism, one would generally ensure that a significant period of time did not expire between the time of each delivery, such that the imexon and/or derivative thereof/lipid composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the imexon and/or derivative thereof/lipid composition. In other aspects, one or more agents may be administered within of from about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, to about 48 hours or more prior to and/or after administering the imexon and/or derivative thereof/lipid composition. In certain other embodiments, an agent may be administered within of from about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20, to about 21 days prior to and/or after administering the imexon and/or derivative thereof/lipid composition. In some situations, it may be desirable to extend the time period for treatment significantly, however, where several weeks (e.g., about 1, about 2, about 3, about 4, about 5, about 6, about 7 or about 8 weeks or more) lapse between the respective administrations.

Various combination regimens of the imexon and/or derivative thereof/lipid composition and one or more agents may be employed. Non-limiting examples of such combinations are shown below, wherein an imexon and/or a derivative thereof composition of the invention is “A” and an agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the composition of the present invention to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy.

Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP 16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat, for example, chronic leukemia, non-Hodgkin's lymphoma, Hodgkin's disease, multiple myeloma, and particular cancers of the breast, lung, and ovary. An alkylating agent, may include, but is not limited to, a nitrogen mustard, an ethylenimene, a methylmelamine, an alkyl sulfonate, a nitrosourea or a triazines.

They include but are not limited to: busulfan, chlorambucil, cisplatin, cyclophosphamide (cytoxan), dacarbazine, ifosfamide, mechlorethamine (mustargen), and melphalan. In specific aspects, troglitazaone can be used to treat cancer in combination with any one or more of these alkylating agents, some of which are discussed below.

i. Nitrogen Mustards

A nitrogen mustard may be, but is not limited to, mechlorethamine (HN₂), which is used for Hodgkin's disease and non-Hodgkin's lymphomas; cyclophosphamide and/or ifosfamide, which are used in treating such cancers as acute or chronic lymphocytic leukemias, Hodgkin's disease, non-Hodgkin's lymphomas, multiple myeloma, neuroblastoma, breast, ovary, lung, Wilm's tumor, cervix testis and soft tissue sarcomas; melphalan (L-sarcolysin), which has been used to treat such cancers as multiple myeloma, breast and ovary; and chlorambucil, which has been used to treat diseases such as, for example, chronic lymphatic (lymphocytic) leukemia, malignant lymphomas including lymphosarcoma, giant follicular lymphoma, Hodgkin's disease and non-Hodgkin's lymphomas.

a. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. For example, after a single oral doses of about 0.6 mg/kg to about 1.2 mg/kg, peak plasma chlorambucil levels are reached within one hour and the terminal half-life of the parent drug is estimated at about 1.5 hours. About 0.1 mg/kg/day to about 0.2 mg/kg/day or about 36 mg/m²/day to about 6 mg/m²/day or alternatively about 0.4 mg/kg may be used for antineoplastic treatment. Chlorambucil is not curative by itself but may produce clinically useful palliation.

b. Cyclophosphamide

Cyclophosphamide is 2H-1,3,2-Oxazaphosphorin-2-amine, N,N-bis(2-chloroethyl)tetrahydro-,2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal trace tolerated well and effective by the oral and parental routes and does not cause local vesication, necrosis, phlebitis or even pain.

Suitable oral doses for adults include, for example, about 1 mg/kg/day to about 5 mg/kg/day (usually in combination), depending upon gastrointestinal tolerance; or about 1 mg/kg/day to about 2 mg/kg/day; intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. In some aspects, a dose of about 250 mg/kg/day may be administered as an antineoplastic. Because of gastrointestinal adverse effects, the intravenous route is preferred for loading. During maintenance, a leukocyte count of about 3000/ mm³ to 4000/ mm³ usually is desired. The drug also sometimes is administered intramuscularly, by infiltration or into body cavities. It is available in dosage forms for injection of about 100 mg, about 200 mg and about 500 mg, and tablets of about 25 mg and about 50 mg.

c. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of about 2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of about 0.2 mg/kg daily for five days as a single course. Courses are repeated about every four to five weeks depending upon hematologic tolerance (Smith and Rutledge, 1975; Young et al., 1978). Alternatively in certain embodiments, the dose of melphalan used could be as low as about 0.05 mg/kg/day or as high as about 3 mg/kg/day or greater.

ii. Ethylenimenes and Methymelamines

An ethylenimene and/or a methylmelamine include, but are not limited to, hexamethylmelamine, used to treat ovary cancer; and thiotepa, which has been used to treat bladder, breast and ovary cancer.

iii. Alkyl Sulfonates

An alkyl sulfonate includes but is not limited to such drugs as busulfan, which has been used to treat chronic granulocytic leukemia.

Busulfan (also known as myleran) is a bifunctional alkylating agent. Busulfan is known chemically as 1,4-butanediol dimethanesulfonate. Busulfan is available in tablet form for oral administration, wherein for example, each scored tablet contains about 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. Busulfan has been shown to be superior to splenic irradiation with respect to survival times and maintenance of hemoglobin levels, and to be equivalent to irradiation at controlling splenomegaly.

iv. Nitrosourea

Nitrosureas, like alkylating agents, inhibit DNA repair proteins. They are used to treat non-Hodgkin's lymphomas, multiple myeloma, malignant melanoma, in addition to brain tumors. A nitrosourea include but is not limited to a carmustine (BCNU), a lomustine (CCNU), a semustine (methyl-CCNU) or a streptozocin. Semustine has been used in such cancers as a primary brain tumor, a stomach or a colon cancer. Stroptozocin has been used to treat diseases such as a malignant pancreatic insulinoma or a malignaint carcinoid. Streptozocin has beeen used to treat such cancers as a malignant melanoma, Hodgkin's disease and soft tissue sarcomas.

a. Carmustine

Carmustine (sterile carmustine) is one of the nitrosoureas used in the treatment of certain neoplastic diseases. It is 1,3 bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has been used in treating such cancers as a multiple myeloma or a malignant melanoma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

Sterile carmustine is commonly available in 100 mg single dose vials of lyophilized material. The recommended dose of carmustine as a single agent in previously untreated patients is about 150 mg/m² to about 200 mg/m² intravenously every 6 weeks. This may be given as a single dose or divided into daily injections such as about 75 mg/m² to about 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention, for example about 10 mg/m², about 20 mg/m², about 30 mg/m², about 40 mg/m², about 50 mg/m², about 60 mg/m², about 70 mg/m², about 80 mg/m², about 90 mg/m² to about 100 mg/m².

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (about 0.05 mg/mL) and in absolute alcohol (about 70 mg/mL). Lomustine is relatively insoluble in water (less than about 0.05 mg/mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from about 30 mg/m² to 100 mg/m², about half of the radioactivity given was excreted in the form of degradation products within 24 hours. The serum half-life of the metabolites ranges from about 16 hours to about 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. Lomustine has been used to treat such cancers as small-cell lung cancer. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is about 130 mg/m² as a single oral dose every 6 weeks. In individuals with compromised bone marrow function, the dose should be reduced to about 100 mg/m² every 6 weeks. When lomustine is used in combination with other myelosuppressive drugs, the doses should be adjusted accordingly. It is understood that other doses may be used for example, about 20 mg/m², about 30mg/m², about 40 mg/m², about 50 mg/m², about 60 mg/m², about 70 mg/m², about 80 mg/m², about 90 mg/m², about 100 mg/m² to about 120 mg/m².

c. Triazine

A triazine include but is not limited to such drugs as a dacabazine (DTIC; dimethyltriazenoimidaz olecarboxamide), used in the treatment of such cancers as a malignant melanoma, Hodgkin's disease and a soft-tissue sarcoma.

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

i. Folic Acid Analogs

Folic acid analogs include but are not limited to compounds such as methotrexate (amethopterin), which has been used in the treatment of cancers such as acute lymphocytic leukemia, choriocarcinoma, mycosis fungoides, breast, head and neck, lung and osteogenic sarcoma.

ii. Pyrimidine Analogs

Pyrimidine analogs include such compounds as cytarabine (cytosine arabinoside), 5-fluorouracil (fluouracil; 5-FU) and floxuridine (fluorode-oxyuridine; FudR). Cytarabine has been used in the treatment of cancers such as acute granulocytic leukemia and acute lymphocytic leukemias. Floxuridine and 5-fluorouracil have been used in the treatment of cancers such as breast, colon, stomach, pancreas, ovary, head and neck, urinary bladder and topical premalignant skin lesions.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

iii. Purine Analogs and Related Inhibitors

Purine analogs and related compounds include, but are not limited to, mercaptopurine (6-mercaptopurine; 6-MP), thioguanine (6-thioguanine; TG) and pentostatin (2-deoxycoformycin). Mercaptopurine has been used in acute lymphocytic, acute granulocytic and chronic granulocytic leukemias. Thrioguanine has been used in the treatment of such cancers as acute granulocytic leukemia, acute lymphocytic leukemia and chronic lymphocytic leukemia. Pentostatin has been used in such cancers as hairy cell leukemias, mycosis fungoides and chronic lymphocytic leukemia.

c. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

i. Mitotic Inhibitors

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

a. Epipodophyllotoxins

Epipodophyllotoxins include such compounds as teniposide and VP16, VP16 is also known as etoposide and is used primarily for treatment of testicular tumors, in combination with bleomycin and cisplatin, and in combination with cisplatin for small-cell carcinoma of the lung. Teniposide and VP16 are also active against cancers such as testis, other lung cancer, Hodgkin's disease, non-Hodgkin's lymphomas, acute granulocytic leukemia, acute nonlymphocytic leukemia, carcinoma of the breast, and Kaposi's sarcoma associated with acquired immunodeficiency syndrome (AIDS).

VP16 is available as a solution (e.g., 20 mg/ml) for intravenous administration and as 50 mg, liquid-filled capsules for oral use. For small-cell carcinoma of the lung, the intravenous dose (in combination therapy) is can be as much as about 100 mg/m² or as little as about 2 mg/m², routinely about 35 mg/m², daily for about 4 days, to about 50 mg/m², daily for about 5 days have also been used. When given orally, the dose should be doubled. Hence the doses for small cell lung carcinoma may be as high as about 200 mg/m² to about 250 mg/m². The intravenous dose for testicular cancer (in combination therapy) is about 50 mg/m² to about 100 mg/m² daily for about 5 days, or about 100 mg/m² on alternate days, for three doses. Cycles of therapy are usually repeated about every 3 to 4 weeks. The drug should be administered slowly (e.g. about 30 minutes to about 60 minutes) as an infusion in order to avoid hypotension and bronchospasm, which are probably due to the solvents used in the formulation.

b. Taxoids

Taxoids are a class of related compounds isolated from the bark of the ash tree, Taxus brevifolia. Taxoids include but are not limited to compounds such as docetaxel and paclitaxel.

Paclitaxel binds to tubulin (at a site distinct from that used by the vinca alkaloids) and promotes the assembly of microtubules. Paclitaxel is being evaluated clinically; it has activity against malignant melanoma and carcinoma of the ovary. In certain aspects, maximal doses are about 30 mg/m² per day for about 5 days or about 210 mg/m² to about 250 mg/m² given once about every 3 weeks.

c. Vinca Alkaloids

Vinca alkaloids are a type of plant alkaloid identified to have pharmaceutical activity. They include such compounds as vinblastine (VLB) and vincristine.

1. Vinblastine

Vinblastine is an example of a plant alkaloid that can be used for the treatment of cancer and precancer. When cells are incubated with vinblastine, dissolution of the mnicrotubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. When the drug is given intravenously; special precautions must be taken against subcutaneous extravasation, since this may cause painful irritation and ulceration. The drug should not be injected into an extremity with impaired circulation. After a single dose of 0.3 mg/kg of body weight myelosuppression reaches its maximum in about 7 days to about 10 days. If a moderate level of leukopenia (approximately 3000 cells/mm³) is not attained, the weekly dose may be increased gradually by increments of about 0.05 mg/kg of body weight. In regimens designed to cure testicular cancer, vinblastine is used in doses of about 0.3 mg/kg about every 3 weeks irrespective of blood cell counts or toxicity.

An important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, testis cancer, neuroblastoma, and Letterer-Siwe disease (histiocytosis X), as well as in carcinoma of the breast and choriocarcinoma in women.

Doses of about 0.1 mg/kg to about 0.3 mg/kg can be administered or about 1.5 mg/m² to about 2 mg/m² can also be administered. Alternatively, about 0.1 mg/m², about 0.12 mg/m², about 0.14 mg/m², about 0.15 mg/m², about 0.2 mg/m², about 0.25 mg/m², about 0.5 mg/m², about 1.0 mg/m², about 1.2 mg/m², about 1.4 mg/m², about 1.5 mg/m², about 2.0 mg/m², about 2.5 mg/m², about 5.0 mg/m², about 6 mg/m², about 8 mg/m², about 9 mg/m², about 10 mg/m², to about 20 mg/m², can be given.

2. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is about 0.4 mM.

Vinblastine and vincristine bind to plasma proteins. They are extensively concentrated in platelets and to a lesser extent in leukocytes and erythrocytes. Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than about 3 mg/dl (about 50 mM).

Vincristine sulfate is available as a solution (e.g., 1 mg/ml) for intravenous injection. Vincristine used together with corticosteroids is presently the treatment of choice to induce remissions in childhood leukemia; the optimal dosages for these drugs appear to be vincristine, intravenously, about 2 mg/m² of body-surface area, weekly; and prednisone, orally, about 40 mg/m², daily. Adult patients with Hodgkin's disease or non-Hodgkin's lymphomas usually receive vincristine as a part of a complex protocol. When used in the MOPP regimen, the recommended dose of vincristine is about 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, small cell lung, and carcinomas of the breast, bladder, and the male and female reproductive systems.

Doses of vincristine include about 0.01 mg/kg to about 0.03 mg/kg or about 0.4 mg/m² to about 1.4 mg/m² can be administered or about 1.5 mg/m² to about 2 mg/m² can also be administered. Alternatively, in certain embodiments, about 0.02 mg/m², about 0.05 mg/m², about 0.06 mg/m², about 0.07 mg/m², about 0.08 mg/m², about 0.1 mg/m², about 0.12 mg/m², about 0.14 mg/m², about 0.15 mg/m², about 0.2 mg/m², about 0,25 mg/m² can be given as a constant intavenous infusion.

d. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include, but are not limited to, bleomycin, dactinomycin, daunorubicin, doxorubicin (Adriamycin), plicamycin (mithramycin) and idarubicin. Widely used in clinical setting for the treatment of neoplasms these compounds generally are administered through intravenous bolus injections or orally.

1. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of diseases including ovarian, endometrial and breast tumors, bronchogenic oat-cell carcinoma, non-small cell lung carcinoma, stomach, genitourinary, thyroid, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma, soft tissue sarcoma, Ewing's sarcoma, rhabdomyosarcoma and acute lymphocytic leukemia. It is an alternative drug for the treatment of other diseases such as islet cell, cervical, testicular and adrenocortical cancers. It is also an immunosuppressant.

Doxorubicin is absorbed poorly and is preferably administered intravenously. The pharmacokinetics are multicompartmental. Distribution phases have half-lives of 12 minutes and 3.3 hours. The elimination half-life is about 30 hours, with about 40% to about 50% secreted into the bile. Most of the remainder is metabolized in the liver, partly to an active metabolite (doxorubicinol), but a few percent is excreted into the urine. In the presence of liver impairment, the dose should be reduced.

In certain embodiments, appropriate intravenous doses are, adult, about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by about 50% if the serum bilirubin lies between about 1.2 mg/dL and about 3 mg/dL and by about 75% if above about 3 mg/dL. The lifetime total dose should not exceed about 550 mg/m² in patients with normal heart function and about 400 mg/m² in persons having received mediastinal irradiation. In certain embodiments, and alternative dose regiment may comprise about 30 mg/m² on each of 3 consecutive days, repeated about every 4 week. Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin (daunomycin; rubidomycin) intercalates into DNA, blocks DAN-directed RNA polymerase and inhibits DNA synthesis. It can prevent cell division in doses that do not interfere with nucleic acid synthesis.

In combination with other drugs it is often included in the first-choice chemotherapy of diseases such as, for example, acute granulocytic leukemia, acute myelocytic leukemia in adults (for induction of remission), acute lymphocytic leukemia and the acute phase of chronic myelocytic leukemia. Oral absorption is poor, and it preferably given by other methods (e.g., intravenously). The half-life of distribution is 45 minutes and of elimination, about 19 hours. The half-life of its active metabolite, daunorubicinol, is about 27 hours. Daunorubicin is metabolized mostly in the liver and also secreted into the bile (about 40%). Dosage must be reduced in liver or renal insufficiencies.

Generally, suitable intravenous doses are (base equivalent): adult, younger than 60 years, about 45 Mg/m²/day (about 30 mg/m² for patients older than 60 year.) for about 1 day, about 2 days or about 3 days about every 3 weeks or 4 weeks or about 0.8 mg/kg/day for about 3 days, about 4 days, about 5 days to about 6 days about every 3 weeks or about 4 weeks; no more than about 550 mg/m² should be given in a lifetime, except only about 450 mg/m² if there has been chest irradiation; children, about 25 mg/m² once a week unless the age is less than 2 years. or the body surface less than about 0.5 m, in which case the weight-based adult schedule is used. It is available in injectable dosage forms (base equivalent) of about 20 mg (as the base equivalent to about 21.4 mg of the hydrochloride). Exemplary doses may be about 10 mg/m², about 20 mg/m², about 30 mg/m², about 50 mg/m², about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

3. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed. Mitomycin has been used in tumors such as stomach, cervix, colon, breast, pancreas, bladder and head and neck.

In humans, mitomycin is rapidly cleared from the serum after intravenous administration. Time required to reduce the serum concentration by about 50% after a 30 mg, bolus injection is 17 minutes. After injection of 30 mg, 20 mg, or 10 mg I.V., the maximal serum concentrations were 2.4 mg/mL, 1.7 mg/mL, and 0.52 mg/mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is often a component of first-choice combinations for treatment of diseases such as, for example, choriocarcinoma, embryonal rhabdomyosarcoma, testicular tumor, Kaposi's sarcoma and Wilms' tumor. Tumors that fail to respond to systemic treatment sometimes respond to local perfusion. Dactinomycin potentiates radiotherapy. It is a secondary (efferent) immunosuppressive.

In certain specific aspects, actinomycin D is used in combination with agents such as, for example, primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hours. The drug does not pass the blood-brain barrier. Actinomycin D is supplied as a lyophilized powder (0/5 mg in each vial). The usual daily dose is about 10 mg/kg to about 15 mg/kg; this is given intravenously for about 5 days; if no manifestations of toxicity are encountered, additional courses may be given at intervals of about 3 weeks to about 4 weeks. Daily injections of about 100 mg to about 400 mg have been given to children for about 10 days to about 14 days; in other regimens, about 3 mg/kg to about 6 mg/kg, for a total of about 125 mg/kg, and weekly maintenance doses of about 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be about 100 mg/m², about 150 mg/m², about 175 mg/m², about 200 mg/m², about 225 mg/m², about 250 mg/m², about 275 mg/m², about 300 mg/m², about 350 mg/m², about 400 mg/m², about 425 mg/m², about 450 mg/m², about 475 mg/m², to about 500 mg/m².

5. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of greater than about 35 mL per minute, the serum or plasma terminal elimination half-life of bleomycin is approximately 115 minutes. In patients with a creatinine clearance of less than about 35 mL per minute, the plasma or serum terminal elimination half-life increases exponentially as the creatinine clearance decreases. In humans, about 60% to about 70% of an administered dose is recovered in the urine as active bleomycin. In specific embodiments, bleomycin may be given by the intramuscular, intravenous, or subcutaneous routes. It is freely soluble in water. Because of the possibility of an anaphylactoid reaction, lymphoma patients should be treated with two units or less for the first two doses. If no acute reaction occurs, then the regular dosage schedule may be followed.

In preferred aspects, bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), esophagus, lung and genitourinary tract, Hodgkin's disease, non-Hodgkin's lymphoma, skin, penis, cervix, and vulva. It has also been used in the treatment of lymphomas and testicular carcinoma.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Hormones and Antagonists

Hormonal therapy may also be used in conjunction with the present invention and/or in combination with any other cancer therapy or agent(s). The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

i. Adrenocorticosteroids

Corticosteroid hormones are useful in treating some types of cancer (e.g., non-Hodgkin's lymphoma, acute and chronic lymphocytic leukemias, breast cancer, and multiple myeloma). Though these hormones have been used in the treatment of many non-cancer conditions, they are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

ii. Other Hormones and Antagonists

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

e. Miscellaneous Agents

Some chemotherapy agents do not qualify into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they are included within the compositions and methods of the present invention for use in combination therapies.

i. Platinum Coordination Complexes

Platinum coordination complexes include such compounds as carboplatin and cisplatin (cis-DDP). Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes, such as for example, intravenous, subcutaneous, intratumoral or intraperitoneal injection. Cisplatin can be used alone or in combination with other agents, with efficacious doses used in clinical applications of about 15 mg/m² to about 20 mg/m² for 5 days every three weeks for a total of three courses being contemplated in certain embodiments. Doses may be, for example, about 0.50 mg/m², about 1.0 mg/m², about 1.50 mg/m², about 1.75 mg/m², about 2.0 mg/m², about 3.0 mg/m², about 4.0 mg/m², about 5.0 mg/m², to about 10 mg/m².

ii. Other Agents

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

2. Radiotherapeutic Agents

Radiotherapeutic agents include radiation and waves that induce DNA damage for example, γ-irradiation, X-rays, proton beams, UV-irradiation, microwaves, electronic emissions, radioisotopes, and the like. Therapy may be achieved by irradiating the localized tumor site with the above described forms of radiations. It is most likely that all of these agents effect a broad range of damage DNA, on the precursors of DNA, the replication and repair of DNA, and the assembly and maintenance of chromosomes.

Radiotherapeutic agents and methods of administration, dosages, etc. are well known to those of skill in the art, and may be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes, for example, preventative, diagnostic or staging, curative and palliative surgery. Surgery, and in particular a curative surgery, may be used in conjunction with other therapies, such as the present invention and one or more other agents.

Curative surgery includes resection in which all or part of cancerous tissue is physically removed, excised and/or destroyed. It is further contemplated that surgery may remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity may be formed in the body.

Further treatment of the tumor or area of surgery may be accomplished by perfusion, direct injection or local application of the area with an additional anti-cancer agent. Such treatment may be repeated, for example, about every 1, about every 2, about every 3, about every 4, about every 5, about every 6, or about every 7 days, or about every 1, about every 2, about every 3, about every 4, or about every 5 weeks or about every 1, about every 2, about every 3, about every 4, about every 5, about every 6, about every 7, about every 8, about every 9, about every 10, about every 11, or about every 12 months. These treatments may be of varying dosages as well.

4. Immunotherapeutic Agents

An immunotherapeutic agent generally relies on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (e.g., a chemotherapeutic, a radionuclide, a ricin A chain, a cholera toxin, a pertussis toxin, etc.) and serve merely as a targeting agent. Such antibody conjugates are called immunotoxins, and are well known in the art (see U.S. Pat. No. 5,686,072, 5,578,706, 4,792,447, 5,045,451, 4,664,911, and 5,767,072, each incorporated herein by reference) Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

a. Immune Stimulators

In a specific aspect of immunotherapy is to use an immune stimulating molecule as an agent, or more preferably in conjunction with another agent, such as for example, a cytokines such as for example IL-2, IL-4, IL-12, GM-CSF, tumor necrosis factor; interferons alpha, beta, and gamma; F42K and other cytokine analogs; a chemokine such as for example MIP-1, MIP-1beta, MCP-1, RANTES, IL-8; or a growth factor such as for example FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

Another cytokine specifically contemplate is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

b. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. For example, human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers (Bajorin et al., 1988).

C. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

d. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

5. Genetic Therapy Agents

A tumor cell resistance to agents, such as chemotherapeutic and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with the imexon, and/or a derivative thereof/lipid composition and/or other agents.

a. Inducers of Cellular Proliferation

In one embodiment of the present invention, it is contemplated that anti-sense mRNA directed to a particular inducer of cellular proliferation is used to prevent expression of the inducer of cellular proliferation. The proteins that induce cellular proliferation further fall into various categories dependent on function. The commonality of all of these proteins is their ability to regulate cellular proliferation.

For example, a form of PDGF, the sis oncogene, is a secreted growth factor. Oncogenes rarely arise from genes encoding growth factors, and at the present, sis is the only known naturally-occurring oncogenic growth factor.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

Other proteins such as Jun, Fos and Myc are proteins that directly exert their effects on nuclear functions as transcription factors.

b. Inhibitors of Cellular Proliferation

In certain embodiment, the restoration of the activity of an inhibitor of cellular proliferation through a genetic construct is contemplated. Tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p₁₆^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

In certain embodiments, it is contemplated that genetic constructs that stimulate apoptosis will be used to promote the death of diseased or undesired tissue. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

6. Other Biological Agents

It is contemplated that other agents may be used in combination with the present invention to improve the therapeutic efficacy of treatment. These additional agents include, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents such as for example, hyperthermia.

It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population.

In other embodiments, cytostatic or differentiation agents can be used in combination with the present invention to improve the anti-hyerproliferative efficacy of the treatments.

Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as, for example, the antibody C225, could be used in combination with the present invention to improve the treatment efficacy.

Another form of therapy for use in conjunction with the present invention and/or other agent(s) includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

D. Pharmaceutical Preparations

Pharmaceutical aqueous compositions of the present invention comprise an effective amount of one or more imexon and/or derivatives thereof, lipid, and/or additional agent dissolved or dispersed in a pharmaceutically acceptable carrier or aqueous medium. The phrases “pharmaceutically or pharmacologically acceptable” refers to molecular entities and compositions that do not produce an adverse, allergic or other untoward reaction when administered to a human. As used herein, “pharmaceutically acceptable carrier” includes any and all solvents, dispersion media, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The actual dosage amount of a composition of the present invention administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

The present invention can be administered intravenously, intradermnally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally and/or using aerosol, injection, infusion, continuous infusion, localized perfusion bathing target cells directly or via a catheter and/or lavage. For example, the imexon and/or derivative thereof, lipid, and/or additional agent may be formulated for parenteral administration, e.g., formulated for injection via the intravenous, intramuscular or sub-cutaneous routes, though other routes such aerosol administration may be used. The preparation of an aqueous composition that contains at least one imexon and/or derivative thereof, lipid, and/or additional agent as an active ingredient will be known to those of skill in the art in light of the present disclosure, as exemplified by Remington's Pharmaceutical Sciences, 16th Ed. Mack Publishing Company, 1980, incorporated herein by reference. Moreover, for human administration, it will be understood that preparations should meet sterility, pyrogenicity, general safety and purity standards as required by FDA Office of Biological Standards.

Typically, such compositions can be prepared as injectables, either as liquid solutions or suspensions; solid forms suitable for preparing solutions or suspensions upon the addition of a liquid prior to injection can also be prepared; and the preparations can also be emulsified. The compositions will be sterile, be fluid to the extent that easy syringability exists, stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

Although it is most preferred that compositions of imexon and/or derivatives thereof, lipid, and/or additional agent be prepared in sterile water containing other non-active ingredients, made suitable for injection, solutions of such active ingredients can also be prepared in water suitably mixed with a surfactant, such as hydroxypropylcellulose, if desired. Dispersions can also be prepared in liquid polyethylene glycols, and mixtures thereof and in oils. The carrier can also be a solvent or dispersion medium containing, for example, water, ethanol, polyol (for example, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Upon formulation, solutions will be administered in a manner compatible with the dosage formulation and in such amount as is therapeutically effective. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

It is particularly contemplated that suitable pharmaceutical compositions of imexon and/or derivatives thereof, lipid, and/or additional agent will generally comprise, but are not limited to, from about 10 to about 100 mg of the desired imexon and/or derivative thereof, lipid, and/or additional agent admixed with an acceptable pharmaceutical diluent or excipient, such as a sterile aqueous solution, to give a final concentration of about 0.25 to about 2.5 mg/ml with respect to the conjugate, in, for example, 0.15M NaCl aqueous solution at pH 7.5 to 9.0. The preparations may be stored frozen at −10° C. to −70° C. for at least 1 year.

E. Kits

Any of the compositions described herein may be comprised in a kit. In a non-limiting example, an imexon and/or a derivative thereof, lipid, and/or additional agent, may be comprised in a kit. The kits will thus comprise, in suitable container means, an imexon and/or a derivative thereof and a lipid, and/or an additional agent of the present invention.

The kits may comprise a suitably aliquoted imexon and/or derivative thereof, lipid and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the imexon and/or derivative thereof, lipid, additional agent, and any other reagent containers in close confinement for commercial sale. Such containers may include injection or blow-molded plastic containers into which the desired vials are retained.

F. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The synthesis of 2-cyanoaziridine-1-carboxamide derivatives of imexon have been described in Iyengar et al., 1999. The synthesis of various derivatives of imexon has also been described in Examples 1-19 of WO 99/00120, and the relevant text of these examples are incorporated herein below.

### EXAMPLE 1

### 2-Cyanoaziridine-1-(N-methyl) carboxainide

To an ice-cold mixture of 2-cyanoaziridine and toluene was added an ice-cold solution of methyl isocyanate (1.05 equivalents) in toluene at a rate to keep the temperature below 5° C. The mixture was stirred for one hour in an ice bath and then placed in a refrigerator overnight. The resulting precipitate was collected, washed with toluene, and dried under vacuum to give a 94% yield of the title compound as a solid with m.p. 98-100° C.: MS (EI) 125 (M+); ¹H NMR (CDCl₃, TMS) 2.47 (d, 1, J=3 Hz), 2.57 (d, 1, J=6 Hz) 2.8 (d, 3, J=5 Hz), 3.05 (2d, 1, J=6 Hz, 3 Hz) 6.18 (s, 1, NH).

### EXAMPLE 2

### 2-Cyanoaziridine-1-(N-ethyl) carboxamide

This compound was prepared from 2-cyanoaziridine and ethylisocyanate in 63% yield by the procedure described in Example 1. It had m.p. 58-62° C.: MS (EI) 139 (M⁺) ¹H NMR (CDCl₃, TMS) 1.1 (t, 3, J=6 Hz), 2.4 (d, 1, J=3 Hz) 2.50 (d, 1, J=6 Hz), 2.97 (2d, 1, J=6 Hz, 3 Hz) 3.3 (q, 2, J=6 MZ), 6.1 (s, 1, NH).

### EXAMPLE 3

### 2-Cyanoaziridine-1-(N-butyl) carboxamide

This compound was prepared from 2-cyanoaziridine and n-butylisocyanate in 92% yield by the procedure described in Example 1. It had m.p. 32-34° C.: ¹H NMR (CDCl₃, TMS) 1.0 (t, 3), 1.2-1.5 (m, 2), 1.6-2.1 (m. 2), 2.5 (d, 1, J=3 Hz), 2.55 (d, 1, J=6 Hz), 3.19 (2d, 1, J=6 Hz, 3 Hz), 3.6 (m, 2), 5.8 (s, 1, NH).

### EXAMPLE 4

### 2-Cyanoaziridine-1-(N-t-butyl) carboxamide

This compound was prepared from 2-cyanoaziridine and 1-butylisocyanate in 81% yield by the procedure described in Example 1. It had m.p. 46-48° C.: ¹H NMR (DMSO-d₆ TMS) 1.4 (s, 9), 2.84 (d, 1, J=3 Hz), 2.86 (d, 1, J=6 Hz), 3.0 (2d, 1, J=6 Hz, 3 Hz) 5.8 (br. s, NH).

### EXAMPLE 5

### 2-Cyanoaziridine-1-(N-cyclohexyl) carboxamide

This compound was prepared from 2-cyanoaziridine and cyclohexylisocyanate in 64% yield by the procedure described in Example 1. It had m.p. 98-102° C.: ¹H NMR (CDCl₃, TMS) 1.2-1.5 (m, 5), 1.6-2.1 (m, 5), 2.45 (d, 1, J=3 Hz), 2.53 (d, 1, J=6 Hz), 3.015 (2d, 1, J=6Hz, 3 Hz), 3.6 (m, 1), 5.8 (s, 1, NH).

### EXAMPLE 6

### 2-Cyanoaziridine-1-(N-benzyl) carboxamide

Method A. This compound was prepared from 2-cyanoaziridine and benzylisocyanate in 25% yield by the procedure described in Example 1. It had m.p. 42-44° C.: ¹H NMR (CDCl₃, TMS), 2.36 (d, 1, J=3 Hz), 2.46 (d, 1, J=6 Hz), 2.93 (2d, 1, J=6 Hz, 3 Hz), 4.4 (d, 2), 6.8 (s, 1, NH), 7.4 (m, 5).

Methods B. A solution of 175 mg of 2-cyanoaziridine and 0.36 mL of triethylamine in 5 mL of THF was cooled and stirred in an ice bath. A solution of 550 mg of 4-Nitrophenyl chloroformate in 2.5 mL of THF was added at a rate that kept the temperature below 10° C. When the addition was complete, the solution was stirred 2 hours at room temperature and then filtered to remove triethylamine hydrochloride. The filtrate was concentrated under reduced pressure and the residual oil was stirred with 5 mL of toluene for 30 minutes. The pale yellow precipitate that formed was washed with toluene (2×5 mL) and dried under vacuum to afford a 33% yield of 4-nitrophenyl 2-cyanoaziridine-1 carboxylate, m.p. 100-104° C.

A mixture of 4-nitrophenyl 2-cyanoaziridine-1-carboxylate and benzylamine (1:1.1 molar ratio) in THF was stirred vigorously at room temperature. The progress of the reaction was monitored by thin-layer chromatography on silica gel with chloroform-methanol (1:9) as solvent. When the starting materials were consumed (about 2 hours), the solution was concentrated under reduced pressure and the title compound was freed from 4-nitrophenol by washing with THF. This procedure gave the title compound with m.p 42-44° C. and a ¹H NMR spectrum identical with that of the itsample described in Method A.

### EXAMPLE 7

### 2-Cyanoaziridine-1-[N-(ethoxycarbonyl)methyl]carboxamide

This compound was prepared from 2-cyanoaziridine and ethyl isocyanatoacetate in 50% yield by the procedure described in Example 1, except that it was colorless oil and did not crystallize on refrigeration. Instead, it was dissolved in cold chloroform and diluted with cold hexane. The mixture was stirred briefly and then the solvent was decanted. The residual title compound had ¹H NMR (CDCI₃, TMS) 1.22 (t, 3, J=7 Hz), 2.49 (d, 1, J=3 Hz), 2.56 (d, 1, J=6 Hz), 3.07 (2d, J=6 Hz, 3 Hz), 3.91 (d, 2), 4.15 (q, 2, J=7 Hz), 6.74 (t, 1, NH).

### EXAMPLE 8

### 2-Cyanoaziridine-1-(N-phenyl)carboxamide

This compound was prepared from 2-cyanoaziridine and phenylisocyanate in 71% yield by the procedure described in Example 1. It had m.p. 88-90° C.: MS (EI) 187(M³⁰); ¹H NMR (DMSO-d₆, TMS) 2.65 (d, 1, J=3 Hz), 2.69 (d, 1, J=6 Hz), 3.57 (2d, 1, J=6 Hz, 3 Hz), 7.05 (t, 1), 7.45 (d, 2), 7.60 (d, 2), 10.2 (s, 1, NH).

### EXAMPLE 9

### 2-Cyanoaziridine-1-[N-(4-fluorophenyl)]carboxamide

This compound was prepared from 2-cyanoaziridine and (4-flourophenyl)isocyanate in 54% yield by the procedure described in Example 1. It had m.p. 99-100° C.: ¹H NMR (DMSO-d₆, TMS) 2.55 (d, 1, J=3 Hz), 2.68 (d, 1, J=6 Hz), 3.20 (2d, 1, J=6 Hz, 3 Hz), 7.0 (d, 2, J=9 Hz), 7.5 (d, 2, J=9 Hz), 10.2 (s, 1, NH).

### EXAMPLE 10

### 2-Cyanoaziridine-1-[N-(4-trifluorophenyl)]carboxamide

This compound was prepared from 2-cyanoaziridine and 4-(trifluorophenyl)isocyanate in 91% yield by the procedure described in Example 1. It had m.p. 166-168° C.: ¹H NMR (DMSO-d₆, TMS) 2.62 (d, 1, J=3 Hz), 2.74 (d, 1, J=6 Hz), 3.32 (2d, 1, J=6 Hz, 3 Hz), 7.54 (d, 2, J=9 Hz), 7.74 (d, 2, J=9 Hz) 10.2 (s, 1, NH).

### EXAMPLE 11

### 2-Cyanoaziridine-1-[N-(4-nitrophenyl)]carboxamide

This compound was prepared from 2-cyanoaziridine and 4-(nitrophenyl)isocyanate in 89% yield by the procedure described in Example 1. It decomposed above 230° C.: ¹H NMR (DMSO-d₆, TMS) 2.77 (d, 1, J=3 Hz), 2.81 (d, 1, J=6 Hz), 3.69 (2d, 1, J=6 Hz, 3 Hz), 7.8 (d, 2, J=9 Hz), 8.2(d, 2, J=9 Hz), 10.8 (s, 1, NH).

### EXAMPLE 12

### 2-Cyanoaziridine-1-[N-(2,4-dichlorophenyl)]carboxamide

This compound was prepared from 2-cyanoaziridine and 2,4-dichlorophenyl)isocyanate in 50% yield by the procedure described in Example 1. It had m.p. 110-114° C.: ¹H NMR (DMSO-d₆, TMS) 2.70 (d, 1, J=3 Hz), 2.71 (d, 1, J=6 Hz), 3.57 (2d, 1, J=6 Hz, 3 Hz), 7.4 (d, 1, J=6 Hz), 7.6 (d, 1, J=6 Hz), 7.7 (s, 1), 10.0 (s, 1, NH).

### EXAMPLE 13

### 2-Cyanoaziridine-1-[N-(3,4-dichlorophenyl)]carboxamide

This compound was prepared from 2-cyanoaziridine and (3,4-dichlorophenyl)isocyanate in 76% yield by the procedure described in Example 1. It had m.p. 132-134° C.: ¹H NMR (DMSO-d₆, TMS) 2.71 (d, 1, J=3 Hz), 2.73 (d, 1, J=6 Hz), 3.62 (2d, 1, J=6 Hz, 3 Hz), 7.5 (2d, 1, J=9 Hz, 3 Hz), 7.6 (d, 1, J=3 Hz), 10.6 (s, 1, NH).

### EXAMPLE 14

### 2-Cyanoaziridine-1-[N-(4-ethoxycarbonylphenyl)]carboxamide

This compound was prepared from 2-cyanoaziridine and ethyl 4-isocyanatobenzoate in 90% yield by the procedure described in Example 1. It had m.p. 162-165° C.: ¹H NMR (DMSO-d₆, TMS) 1.3 (t, 3, J=6 Hz), 2.72 (d, 1, J=3 Hz), 2.76 (d, 1, J=6 Hz), 3.64 (2d, 1, J=6 Hz, 3 Hz), 4.3 (q, 2, J=6 Hz), 7.69 (d, 2, J=9 Hz), 7.73 (d, 2, J=9 Hz), 10.63 (s, 1, NH).

### EXAMPLE 15

### 2-Cyanoaziridine-1-[N-(3-acetylphenyl)]carboxamide

This compound was prepared from 2-cyanoaziridine and (3-acetylphenyl)isocyanate in 74% yield by the procedure described in Example 1. It had m.p. 110-112° C.: ¹H NMR (DMSO-d₆, TMS) 2.6 (s, 3), 2.71 (d, 1, J=3 Hz), 2.74 (d, 1, J=6 Hz), 3.63 (2d, 1, J=6 Hz, 3 Hz), 7.5 (t, 1, J=9 Hz), 7.7 (d, 1, J=9 Hz), 7.85 (d, 1, J=9 Hz), 8.1 (s, 1), 10.5 (s, 1, NH).

### EXAMPLE 16

### 2-Cyanoaziridine-1-[N-(2-acetoxyphenyl)]carboxamide

This compound was prepared from 2-cyanoaziridine and (2-acetoxyphenyl)isocyanate in 10% yield by the procecure described in Example 1. It had m.p. 100-102° C.: ¹H NMR (CDCI₃, TMS) 2.38 (s, 3), 2.55 (br. s, 1), 2.64 (br. s, 1,), 3.20 (br. s, 1), 7.15 (br. s, 2), 7.2-7.6 (br. s, 1), 7.68 (br. s, 1), 7.96 (s, 1, NH). In this spectrum, the expected doublets were not resolved, but appeared as broad singlets.

2-Acetylbenzoic acid azide was prepared by reacting 2-acetoxybenzoyl chloride with sodium azide in acetone and water at 0-5° C. for 24 hours. It had an IR peak at 2245 cm¹ (azide). The crude azide was then heated in benzene at 70-75° C. under nitrogen for 2 hours to give 2-acetoxyphenylisocyanate.

### EXAMPLE 17

### 2-Cyanoaziridine-1-[N-(4-sulfamylphenyl)]carboxamide

2-Cyanoaziridine-1-[N-(4-chlorosulfonyl)]carboxamide was prepared from 2-cyanoaziridine and (4-chlorosulfonylpheny)isocyanate by the procedure described in Example 1. It had m.p. 142-144° C. Without further purification, it was converted by treatment with liquid ammonia into the title compound, in overall yield of 39%: m.p. 170-174° C.; ¹H NMR (DMSO-d₆, TMS) 2.72 (d, 1, J=3 Hz), 2.74 (d, 1, J=6 Hz), 3.7 (2d, 1, J=6 Hz, 3 Hz), 7.26 (s, 2, SO₂NH₂), 7.69 (d, 2), 7.73 (d, 2), 10.6 (s, 1, CONH₂).

### EXAMPLE 18

### 2-Cyanoaziridine-1-[N-(1-naphthyl)]carboxamide

This compound was prepared from 2-cyanoaziridine and 1-naphthylisocyanate in 56% yield by the procedure described in Example 1. It had m.p. 98-100° C.: ¹H NMR (CDCI₃, TMS) 2.6 (br. s, 1,), 2.7 (br. s, 1), 3.2 (br. s, 1), 7.4 (br. s, 1), 7.5 (m, 2), 7.7 (br. s, 2), 7.8 (br. s, 2), 8.1 (s, 1, NH).

In this spectrum, the expected doublets were not resolved and appeared as broad singlets.

### EXAMPLE 19

### 2-Cyanoaziridine-1-[N-(3-pyridyl)]carboxamide

This compound was prepared from 2-cyanoaziridine and 3-pyridylisocyanate by the procedure described in Example 1, except that the solvent was benzene. It had m.p. 205° C. (dec.): ¹H NMR (DMSO-d₆, TMS) 2.72 (d, 1, J=3 Hz), 2.76 (d, 1, J=6 Hz), 3.65 (2d, 1, J=6 Hz, 3 Hz), 7.36 (d, 1), 7.97 (d, 1), 8.27 (d, 1), 8.71 (s, 1, NH), 10.5 (br. 2 , 1, NH).

3-Pyridylisocyanate was prepared from nicotinic acid hydrazide by way of nicotinic acid azide following the literature procedure. (Hyden, et al., 1967.) It had an IR peak at 2250 cm¹ (isocyanate). The overall yield of the title compound was 10%.

### EXAMPLE 20

### Anti-Cancer Activity of Imexon Analogues

To demonstrate the efficacy of liposomal-imexon preparations, three different analogues of imexon (AMP-404, AMP-415 and AMP-416) were tested in a cytotoxic assay against 822R myeloma cells.

Liposomal imexon was prepared by disolving 6 mg of an analogue into 1 ml of t-butanol prior to combining with a liposome. The drugs, Amp 404 DMPC-DMPG, Amp 404 DMPC-PA, Amp 404 DMPC, DMPC empty liposomes, AMP 404 alone, AMP 415 DMPC-DMPG, AMP 415 DMPC-PA, AMP 415 DMPC and AMP 415 alone were tested for cytotoxicity against 8226R (myeloma cell line) using the MTT assay. The 8226R cells were plated at 5×10⁴ cell per well of a flat bottom 96 well plate at 100 ul of cell suspension per well. The liposomal formulations comprising the drug, the liposomal formulations without the drugs and drugs alone were dissolved in CRPMI, culture media, at a concentration 20× the needed concentration per well 100 ul of 20× liposomal formulations or 2× drug alone was added to the appropriate wells. A final 20× dilution was used because there was a 2× dilution in the well and a 10× dilution of lipid to drug. The final concentration per well was 0, 0.1, 5, 10, 50 or 100 ug/ml. Each group was cultured in triplicates. The plates were harvested at days 1, 2 and 3 using the standard MTT assay. The MTT procedure was as follows: spin plates for 5 min at 1000 RPM 4° C.; remove 100 ul supernatant and discard; add 10 ul MTT working solution (5 mg/ml in PBS); incubate at 37° C. for 4 hours; add 150 ul in acid alcohol (0.04 M HCl in isopropanol); incubate at room temperature 5-10 min; mix wells to dissolve blue crystals; and read plates at 570 nm. The results were recorded as % cytotoxicity versus drug concentration.

### EXAMPLE 21

### Antitumor 2-Cyanoaziridine-1-carboxamides

A set of twenty 2-cyanoaziridine-1-carboxamides was synthesized from 2-cyanoaziridine and appropriate isocyanates. These compounds were tested against a panel of tumor cells in culture, and were active against a variety of solid and hematological tumor cells, including strains resistant to doxorubicin and mitoxantrone. Their potencies in these assays correlated with the lipophilicity of substituents. The N-phenyl derivative was more potent and equally effective to imexon, a cyclized 2-cyanoaziridine-1-carboxamide of clinical interest, against cloned fresh human tumors. It is contemplated that one or more of these 2-cyanoaziridine-1-carboxamides derivatives may be used in combination with a lipid and/or another agent.

Relative potencies of the N-substituted 2-cyanoaziridine-1-carboxamides in a panel of tumor cell cultures are compared in Table 3. This panel includes the following lines of human tumor cells: sensitive and imexon-resistant lines of multiple myeloma (Matsuoka et al., 1967); ovarian carcinoma resistant to standard drugs (Hamilton et al., 1984); melanoma with a slow growth fraction (Girard et al., 1973); breast carcinoma including sensitive, doxorubicin-resistant (P-glycoprotein-positive) (Taylor et al., 1991), and mitoxantrone-resistant (P-glycoprotein-negative) (Futscher et al., 1994) lines; sensitive and multidrug-resistant colon carcinoma (Noguchi et al., 1979; Dalton et al., 1988); and multidrug-resistant lung carcinoma (P-glycoprotein-negative) (Girard et al., 1973). It also has sensitive (Goldin et al., 1961) and multidrug-resistant (P-glycoprotein-positive) (Salmon and Hersh, 1994) murine L1210 leukemia cells.

TABLE 3
Antitumor Activities of 2-Cyanoaziridine-1-carboxamides

| | | IC₅₀ against human mouse tumors (μM)^{a} | | | | | | | | | | | |
|---|---|---|---|---|---|---|---|---|---|---|---|---|---|
| | | | | | | L1210 | | | | | | | |
| | | 8226 myeloma | | OVCAR3 | A375 | leukemia | | MCF7 breast | | | WiDr colon | | A-549 |
| No. | R | Sens^{b} | Res^{c} | ovarian | melan | Sens | Mdr | Sens | Dox | mitox | Sens | res | lung |
| Imexon | | 17 | 115 | 640 | 324 | 612 | 477 | >18 | >18 | >18 | >72 | >72 | >72 |
| 1 | H | 288 | 432 | >90 | >90 | >90 | >90 | >54 | >54 | >54 | >280 | >280 | >280 |
| 6 | CH₃ | 26 | 26 | 14 | 17 | 8.7 | 21 | 27 | 17 | 18 | 21 | 23 | 22 |
| 7 | C₂H₅ | 22 | 26 | 14 | 36 | 14 | 14 | 29 | 20 | 10 | 4 | 7 | 18 |
| 8 | n-C₄H₉ | 17 | 17 | 12 | 15 | 12 | 12 | >12 | 6.7 | >12 | 14 | 18 | 18 |
| 9 | t-C₄H₉ | 120 | 216 | 186 | 418 | 132 | 457 | >300 | 190 | 120 | 180 | >300 | 180 |
| 10 | c-C₆H₁₁ | 36 | 98 | 31 | 35 | 7.4 | 16 | 13 | 8.9 | 9.8 | 2.4 | 12 | 10 |
| 11 | CH₂C₆H₅ | 9.0 | 11 | 5.0 | 11 | 3.8 | 10 | 7.4 | 6.0 | 3.4 | 5.3 | 10 | 7.4 |
| 12 | CH₂COOC₂H₅ | 25 | 30 | | 51 | 13 | 13 | 15 | 17 | 11 | 31 | 33 | 24 |
| 13 | C₆H₅ | 5.3 | 8.0 | 6.8 | 11 | 1.2 | 11 | 2.9 | 2.6 | 4.3 | 5.0 | 12 | 9 |
| 14 | 4-FC₆H₄ | 5.0 | 5.9 | 5.9 | 3.4 | 7.3 | 5.9 | 1.9 | 2.4 | 2.7 | 2.1 | 9.0 | 6.2 |
| 15 | 4-CF₃C₆H₄ | 8.2 | 7.8 | 5.9 | 11.8 | 7.8 | 7.1 | 1.5 | 1.4 | 1.3 | 1.3 | 5.8 | 4.3 |
| 16 | 4-O₂NC₆H₄ | 9.1 | 8.6 | 6.5 | 3.9 | 2.6 | 2.6 | 3 | 3 | 3 | 4.3 | 3.7 | 3.9 |
| 17 | 2,4-Cl₂C₆H₃ | 4.7 | 9.4 | 35 | >39 | 207 | 33 | 0.9 | 1.1 | 0.35 | 2.2 | 1.9 | 2.5 |
| 18 | 3,4-Cl₂C₆H₃ | 1.1 | 2.7 | 5.6 | 5.9 | 2.6 | 7.2 | 4.6 | 7.2 | 4.4 | 7.0 | 14 | 7.5 |
| 19 | 4-C₂H₅O₂CC₆H₄ | 3 | 3 | 7.7 | 5.8 | 9.7 | 9.7 | 2.3 | 2.1 | 3.3 | 2.5 | 4.2 | 2.9 |
| 20 | 2-CH₃CO₂C₆H₄ | >39 | >39 | >39 | >39 | >39 | >39 | >16 | >16 | >16 | 40 | >41 | >41 |
| 21 | 3-CH₃COC₆H₄ | 8.7 | 9.6 | 10 | 7.9 | 9.6 | 7.9 | 3.6 | 3.2 | 3.5 | 3.3 | 9 | 5.6 |
| 22 | 4-C₆H₄SO₂NH₂ | >38 | >38 | 188 | 188 | >38 | >38 | >15 | >15 | >15 | >38 | >38 | >38 |
| 23 | 1-naphthyl | 8.0 | 8.9 | 5.9 | 4.2 | 7.2 | 2.5 | 2.0 | 1.7 | 2.0 | 2.4 | 2.5 | 2.5 |
| 24 | 3-pyridyl | 12 | 15 | 10 | 12 | 11 | 16 | 4.2 | 6.6 | 5.4 | 6 | 18 | 16 |
| ^{a}Cytotoxicity against L1210 leukemia was determined by the MTT assay (Alley, Scudiero, Monks, Hursey, Czerwinski, Fine, Abbott, Mayo, Schoemaker, Boyd, “Feasibility of drug screening with panels of human tumor cell lines using | | | | | | | | | | | | | |
| # a microculture tetrazolium assay,” Cancer Res., 48:589-601, 1988 and the SRB assay (Skehan, Strong, Scudiero, Monks, McMahon, Vistica, Warren, Bokesch, Kenney, Boyd, “New colorimetric assay for anticancer-drug screening,” J. Natl. Cancer Inst., 82:1107-1112, 1990) was used for the other tumor cell lines. | | | | | | | | | | | | | |

^{b}Sens indicates a drug-sensitive cell line, and res indicates a resistant cell line.
^{c}This cell line was made specifcially resistent to imexon.

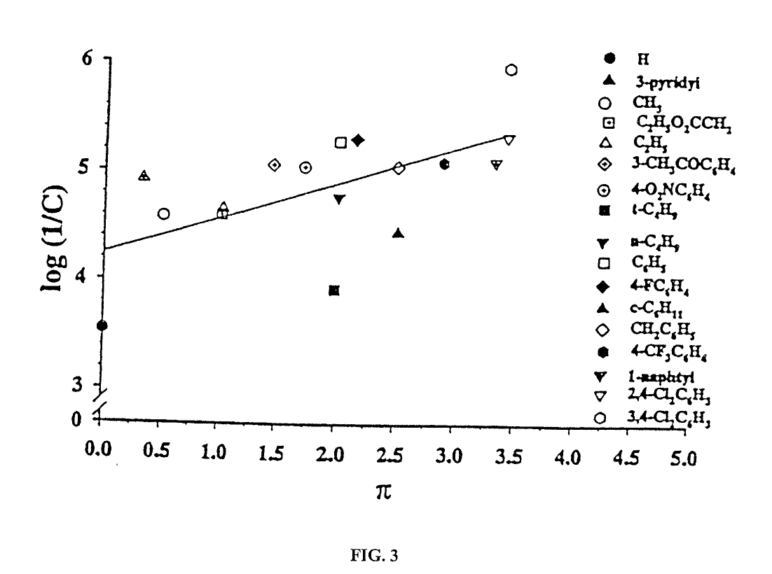

Table 3 shows clearly that imexon is selective for multiple myeloma and that it is more potent than 2-cyanoaziridine-1-carboxamide (1) as expected from previous publications. Addition of lipophilic groups to the amide nitrogen significantly increased the cytotoxicity and decreased the selectivity so that in many cases the compounds were roughly equipotent across the spectrum of tumor cell types. Even the methyl group showed this effect. Among the alkyl-substituted compounds, there was a trend to greater potency as the lipophilicity increased, except that the bulky tert-butyl group has much reduced potency. It may be too large to fit into whatever receptor is critical in cell death. The N-aryl groups, including naphthyl, significantly increase cytotoxicity. Compounds with 4-fluorophenyl (14), 4-nitrophenyl (16), 4-carbethoxyphenyl (19), and naphthyl (23) have IC₅₀ values less than 10 μM for all of the tumor cell types. A statistically significant correlation (99% confidence limit) was found between antitumor potency for sensitive multiple myeloma and the lipophilicity of substituents as represented by their contributions (π) to the octanol-water partition coefficients, using simple linear regression and the program Sigmastat (Sigmastat 1.0 for Windows). The data for this correlation is given in Table 4. For all 17 compounds with definite IC₅₀ values, the equation is log(l/C)=4.25+0.325π (r²=0.39, F=9.44, Esᵢ=0.23, Esᵥ=0.11, where Esᵢ is the standard deviation of the intercept and Esᵥ is the standard deviation of the variable). A plot of this equation is given in FIG. 3. When the bulky tert-butyl-containing analogue 9 is removed, the equation becomes log(l/C)=4.30+0.330π(r²=0.49, n=16, F=13.4, Esᵢ=0.20, Esᵥ=0.09).

TABLE 4
Correlation of Antimyeloma Potency of 2-cyanoaziridine-1-carboxamides
with π^{a}

| Compd | IC₅₀ | Log(1/C) | π |
|---|---|---|---|
| 1 | 288 | 3.54 | 0 |
| 6 | 26 | 4.59 | 0.50 |
| 7 | 22 | 4.66 | 1.02 |
| 8 | 17 | 4.77 | 2.00 |
| 9 | 120 | 3.92 | 1.98 |
| 10 | 36 | 4.44 | 2.51 |
| 11 | 9.0 | 5.05 | 2.50 |
| 12 | 25 | 4.60 | 1.01 |
| 13 | 5.3 | 5.27 | 2.00 |
| 14 | 5.0 | 5.30 | 2.14 |
| 15 | 8.2 | 5.09 | 2.88 |
| 16 | 9.1 | 5.04 | 1.72 |
| 17 | 4.7 | 5.33 | 3.42 |
| 18 | 1.1 | 5.96 | 3.42 |
| 21 | 8.7 | 5.06 | 1.45 |
| 23 | 8.0 | 5.10 | 3.32 |
| 24 | 12 | 4.95 | 0.32 |

^{a}Antitumor data (IC₅₀ against sensitive myeloma) is taken from TABLE 4. π values were taken from Hansch and Leo, Substituent constants for correlation analysis in chemistry and biology, Wiley-Interscience, New York, or calculated using Clog P software, 1979.

Assays of antitumor agents against fresh human tumor-colony-forming cells provide results that predict better the responses obtained in treating cancer patients than those obtained with long established human cell lines (Salmon et al., 1981). For this reason, the N-phenyl derivative 13 was tested against a variety of fresh human tumors obtained at the Arizona Cancer Center using established methodology (Salmon and Hersh, 1994). Table 5 gives a comparison of the potency of 13 with that of imexon in eight different tumor cell types from 44 patients. This table shows that 13 at a concentration of 0.2 μM is about as effective as imexon at 1.0 μM. Both compounds have similiar profiles of activity at these concentrations, with myeloma cells being the most senstive and breast carcinoma, melanoma, and ovarian carcinoma cells also being inhibited significantly. Compound 13 appears to be superior against the sarcoma cells.

TABLE 5
Activity of 13 and Imexon against Cloned Fresh Human Tumors^{a}

| | | | | no. sensitive | | | |
|---|---|---|---|---|---|---|---|
| | | Imexon (μM) | | | 13 (μM) | | |
| Tumor Type | No. tested | 0.1 | 1.0 | 10 | 0.2 | 2.0 | 2.0 |
| Breast | 4 | 0 | 2 | 3 | 2 | 2 | 3 |
| Lung | 2 | 0 | 0 | 2 | 0 | 1 | 2 |
| Melanoma | 10 | 3/5 | 4 | 8 | 3/5 | 8 | 10 |
| Myeloma | 3 | 3 | 3 | 3 | 2/2 | 2/2 | 2/2 |
| Ovary | 12 | 4 | 3/11 | 8 | 3/10 | 10/11 | 10 |
| Other Gynecologic | 4 | 0 | 0 | 1 | 0 | 2 | 3 |
| Stomach | 1 | 0 | 0 | 1 | 1 | 1 | 1 |
| Sarcoma | 8 | 0/2 | 2 | 5 | 2/2 | 4 | 5 |

^{a}Sensitivity is defined as a decrease of 50% or greater in the amount of [³H]thymine taken up by treated tumor-colony-forming cells compared with control tumor-colony-forming cells. The assay followed a standard protocol (Salmon and Hersh, “Sensitivity of multiple myeloma to imexon in the human tumor cloning assay,” J. Natl. Cancer Inst., 86:228-230, 1994.

2-cyanoaziridine-1-carboxamides with alkyl, aryl, and other substituents on the amide nitrogen have greater potency than the N-unsubstituted parent compound against tumor cells in culture. This greater potency correlates with the lipophilicity of the substituents, which indicates that cell penetration may be an important factor in cytotoxicity. Some of them have greater potency and a broader spectrum of activity against tumor cells including activity against resistant tumor cell lines. The one analogue tested in fresh human tumor cells, N-phenyl derivative 13, was similar in activity to imexon and more potent.

Concerning the possibility that N-substituted aziridine-1-carboxamides are cytotoxic because they cyclize to N-substituted imexon analogues, the inventors have made no in vivo studies; however, such cyclizations appear unlikely because a strongly alkaline solution was required for the formation of imexon from 1.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Bangham, et al., “Diffusion of univalent Ions across the Lamellae of Swollen Phospholipids” J. Mol. Biol., 13:238-252, 1965.

Bicker and Fuhse, “Carcinostatic action of 2-cyanaziridines against a sarcoma in rats,” Exp Pathol (Jena). 10(5-6):279-284, 1975.

Bicker, “Immunopharmacological properties of 2-cyanaziridine derivatives,” In Immune Modulation Agents and Their Mechanisms, Fenical and Chirigos (Eds.), Marcel Dekker, New York and Basle, 447-473, 1984.

Bicker, “N-(2-Cyanoethylene)urea—an asparagine analogous cytostatic compound?” Exp. Pathol., 10:106-108, 1975.

Bicker, U. and Hebold, G. “Cancerostatic action of the immune stimulating compound 4-imino-1,3-diazabicyclo[3.1.0]hexane-2-one, BM 06 002 (Proposed INN Name Imexon) on various transplantation tumors,” IRCS Med Sci. Cancer Hematol. Immunol. Allergy Pharmacol., 5:428, 1977.

Canfield et al., Methods in Enzymology, 189:418-422, 1990.

Catalysis in Micellar and Macromolecular Systems, Fendler and Fendler, Academic Press, N.Y., 1975.

Cheng et al, Investigative Radiology, 22:47-55, 1987.

Dalton et al., “Cytogenetic and phenotfpic analysis of a human colon carcinoma cell line resistant to mitoxnntrone,” Cancer Res., 48:1882-1888, 1988.

Deamer and P. Uster, “Liposome Preparation: Methods and Mechanisms,” in Liposomes (M. Ostro, ed.), Marcel Dekker, Inc., New York pp. 27-52, 1983.

Dermer and Ham, Ethyleneimine and other aziridines, Academic Press, New York and London, 425-441, 1969.

Dorr and Von Hoff, Cancer Chemotherapy Handbook, 2nd ed.; Appleton and Lange, Norwalk, Conn., 375-380, 1994.

El-Gorab et al., Biochem. Biophys. Acta, 306:58-66, 1973.

Fraley et al., “Entrapment of a bacterial plasmid in phospholipid vesicles:potential for gene transfer,” Proc. Nat'l. Acac Sci. USA 76:3348-3352, 1979.

Futscher et al., “Analysis of MRP mRNA in mitoxantrone-selected, multidurg resistant human tumor cells,” Biochem. Pharmacol., 47:1601-1606, 1994.

GB Patent No. 2193095

German Patent No. 2,644,820

German Patent No. 2,656,323

German Patent No. 2,727,550

German Patent No. 2,740,248

Ghosh and Bachhawat, “Targeting of liposomes to hepatocytes,” In: Liver Diseases, Targeted Diagnosis and Therapy Using Specific Receptors and Ligands, Wu and Wu (Eds.), Marcel Dekker, New York, pp 87-104, 1991.

Girard et al., “In vitro cultivation of human tumors: establishment of cell lines derived from a series of solid tumors,” J. Natl. Cancer Inst., 51:1417-1423, 1973.

Goldin et al., “Evaluation of antileukemic agents employing advanced leukemia L1210 in mice, IV (Various Compounds),” Cancer Res., 21:27-92, 1961.

Goodridge et al., “Survey of aziridines,” Cancer Chemother. Rep., 26:341-443, 1963.

Gregoriadis and Davis, “Stability of liposomes in vivo and in vitro is promoted by their cholesterol content and the presence of blood cells,” Biochem Biophys Res Commun., 89(4):1287-1293, 1979.

Hamilton et al., “Experimental model systems of ovarian cancer. Applications to the design and evaluation of new treatment approaches,” Semin. Oncol., 11:285-298, 1984.

Heath et al., Chem.Phys.Lipids 40:347-358, 1986.

Hersh et al., “Antitumor effects of imexon combined with myelosuppressive and non-myelosuppressive anticancer agents,” Proc. Am. Assoc. Cancer Res., 36:294, 1995.

Hersh et al., “Antiproliferstive and antitumor activity of the 2-cyanoaziridine compound imexon on tumor cell lines and fresh tumor cells in vitro,” J. Natl. Cancer Inst., 84:1238-1244, 1992.

Hope et al., Biochimica et Biophysica Acta, 812:55-65, 1985.

Hyden and Wilbert, Chem. Ind. (London) 3:1406-1407, 1967.

Iyengar et al., “Novel Antitumor 2-cyanoariridine-1 carboxamides,” J. Med Chem. 42:510-514, 1999.

Jähnich et al., “Chemie von Aziridinecarbonsluren; 2. Mitteilung Eine Eirifach Synthese von 2-Aminopropennitril,” (Chemistry of Aziridinecarboxylic Acids; 2, Note on a Simple Synthesis of 2-Amino ropenenitrile), Synthesis, 1211-1212, 1992.

Kaneda et al., “Introduction and expression of the human insulin gene in adult rat liver,” J Biol Chem., 264(21):12126-12129, 1989.

Kato et al., “Expression of hepatitis B virus surface antigen in adult rat liver.

Co-introduction of DNA and nuclear protein by a simplified liposome method,” J Biol Chem., 266(6):3361-3364, 1991.

Liposome Technology, Gregoriadis, ed., Vol. 1, pp. 29-31, 51-67 and 79-108 (CRC Press Inc., BocaRaton, Fla. 1984.

Martin et al., “Transposition of an antibiotic resistance element in mycobacteria,” Nature, 345(6277):739-743, 1990.

Matsuoka et al., “Production of free light chains of immunoglobulin by a hematopoetic cell line derived from a patient with multiple myelorria,” Proc. Soc. Exp. Biol. Med., 125:1246-1250, 1967.

Mayer et al., Biochimica et Biophysica Acta, 858:161-168, 1986.

Mayhew et al., Biochimica et Biophysica Acta,755:169-174, 1984.

Mayhew et al., Methods in Enzymology, 149:64-77, 1987.

Micksche et al., “Phase I study for a new immunostimulant drug,” BM 06 002, In Immune Modulation and Control of Neoplasia by Adjuvant Therapy, Chirigos (Ed.), Raven Press, New York, 403-413, 1978.

Modem Pharmaceutics, pp. 505-507, Gilbert Baker and Christopher Rhodes, eds., Marcel Dekker Inc., New York, N.Y., 1990.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al., “Liposomes as carriers for in vivo gene transfer and expression,” Methods Enzymol., 149:157-176, 1987.

Noguchi et al., “Characterization of WiDr: a human colon carcinoma cell line,: In vitro, 35:401-407, 1979.

PCT Application U.S. 85/01161

PCT Application U.S. 89/05040

Perales et al., “Gene transfer in vivo: sustained expression and regulation of genes introduced into the liver by receptor-targeted uptake,” Proc. Natl. Acad Sci. USA, 91:4086-4090, 1994.

Remers and Iyengar, “Antitumor Antibiotics,” In Cancer Chemotherapeutic Agent, Foye (Ed.), American Chemical Society, Washington, D.C., 584-592, 1995.

Remington's Pharmaceutical Sciences, 16th Ed. Mack Publishing Company, 1980

Reynolds, “Aziridines,” In Cancer Chemotherapeutic Agents; Foye (Ed.), American Chemical Society, Washington, D.C., 186-197, 1995.

Sagaster et al., “Antitumor activity of imexon,” J. Natl. Cancer Inst., 87:935-936, 1995.

Salmon, S. E. and Hersh, E. M. “Sensitivity of multiple myeloma to imexon in the human tumor cloning assay,” J. Natl. Cancer Inst., 86:228-230, 1994.

Salmon et al., “Evaluation of new anthracycline analogues with the human tumor stem cell assay,” Cancer Chemother. Pharmacol., 6:103-109, 1981.

Schaumann et al., “Use of 2-cyanoaziridine and its derivatives for immunostimulation,” Ger. Offen., 2,736, 296, 1979.

Sigmastat 1.0 for Windows is available from Jandel Scientific, 2591 Kemer Blvd, San Rafael, Calif. 94912-8920.

Smith and Rutledge, “Chemotherapy in advanced ovarian cancer,” Natl. Cancer Inst. Monogr., 42:141-143, 1975.

Szoka and Papahadjopoulos, “Procedure for Preparation of Liposomes With Large Internal Aqueous Space . . . ”, Proc. Natl. Acad. Sci., 1978, 75:4194-4198.

Taylor et al., “Different mechanisms of decreased drug accumulation in doxorubicin and mitoxantrone resistant variants of the MCF7 human breast cancer cell line,” Br. J. Cancer, 63:923-929, 1991.

U.S. Pat. No. 4,083,987

U.S. Pat. No. 4,162,282

U.S. Pat. No. 4,310,505

U.S. Pat. No. 4,533,254

U.S. Pat. No. 4,664,911

U.S. Pat. No. 4,728,575

U.S. Pat. No. 4,728,578

U.S. Pat. No. 4,737,323

U.S. Pat. No. 4,792,447

U.S. Pat. No. 4,921,706

U.S. Pat. No. 5,045,451

U.S. Pat. No. 5,401,511

U.S. Pat. No. 5,578,706

U.S. Pat. No. 5,603,872

U.S. Pat. No. 5,686,072

U.S. Pat. No. 5,767,072

U.S. Pat. No. 5,786,214

U.S. Pat. No. 5,849,718

U.S. Pat. No. 5,871,727

U.S. Pat. No. 5,879,703

U.S. Pat. No. 5,889,155

Wagner et al., Science, 260:1510-1513, 1990.

Wong et al., “Appearance of beta-lactamase activity in animal cells upon liposome-mediated gene transfer,” Gene., 10(2):87-94, 1980.

WO 98/0748

WO 99/00120

WO 99/18933

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Young et al., N. Engl. J. Med., 299:1261-1266, 1978.

What is claimed is:

1. A pharmaceutical composition, comprising an imexon or derivative thereof in combination with one or more lipids.

2. The pharmaceutical composition of claim 1, wherein at least a portion of the lipids comprise micelles.

3. The pharmaceutical composition of claim 1, wherein at least one phospholipid comprises the lipids.

4. The pharmaceutical composition of claim 3, wherein a portion of the lipids comprise liposomes.

5. The pharmaceutical composition of claim 3, wherein the phopholipid is dimyristoyl phosphatidyl choline, dimyristoylphosphatidylglycerol or phosphatidic acid.

6. The pharmaceutical composition of claim 3, comprising a plurality of phospholipids.

7. The pharmaceutical composition of claim 6, wherein the phospholipids comprise dimyristoyl phosphatidyl choline and dimyristoylphosphatidylglycerol.

8. The pharmaceutical composition of claim 7, further defined as comprising dimyristoyl phosphatidyl choline and dimyristoylphosphatidylglycerol in a 7:3 molar ratio.

9. The pharmaceutical composition of claim 6, wherein the plurality of lipids comprise dimyristoyl phosphatidyl choline and phosphatidic acid.

10. The pharmaceutical composition of claim 9, further defined as comprising dimyristoyl phosphatidyl choline and phosphatidic acid in a 7:1 molar ratio.

11. The pharmaceutical composition of claim 1, wherein the imexon or derivative thereof is hydrophobic.

12. The pharmaceutical composition of claim 1, wherein composition comprises imexon.

13. The pharmaceutical composition of claim 1, wherein the composition comprises at least one derivative of imexon.

14. The pharmaceutical composition of claim 13, wherein the derivative of imexon comprises AMP-404.

15. The pharmaceutical composition of claim 11, wherein the derivative of imexon comprises AMP-404.

16. The pharmaceutical composition of claim 11, wherein the derivative of imexon comprises AMP-408.

17. The pharmaceutical composition of claim 11, wherein the derivative of imexon comprises AMP-415.

18. The pharmaceutical composition of claim 11, wherein the derivative of imexon comprises AMP-416.

19. The pharmaceutical composition of claim 11, wherein the derivative of imexon comprises AMP-419.

20. The pharmaceutical composition of claim 11, wherein the derivative of imexon comprises AMP-420.

21. The pharmaceutical composition of claim 11, wherein the derivative of imexon comprises AMP-421.

22. The pharmaceutical composition of claim 11, wherein the derivative of imexon comprises AMP-423.

23. The pharmaceutical composition of claim 11, wherein the derivative of imexon comprises AMP-425.

24. The pharmaceutical composition of claim 13, wherein the derivative of imexon comprises AMP-403, AMP-405, AMP-406, AMP-407, AMP-408, AMP-409, AMP-410, AMP-412, AMP-413, AMP-414, AMP-417, AMP-418, AMP-419, AMP-420, AMP-421, AMP-422, AMP-423, AMP-424 or AMP-425.

25. The pharmaceutical composition of claim 1, further comprising a targeting agent, a diagnostic agent or a second therapeutic agent.

26. The pharmaceutical composition of claim 25, wherein said targeting agent, diagnostic agent or second therapeutic agent is covalently attached to said lipids by a linking moeity.

27. The pharmaceutical composition of claim 25, comprising a second therapeutic agent.

28. The pharmaceutical composition of claim 26, wherein said second therapeutic agent comprises an anticancer agent.

29. The pharmaceutical composition of claim 28, wherein the anticancer agent is chemotherapy agent, a radiotherapy agent, an immune therapy agent, a genetic therapy agent, a hormonal therapy agent or a biological agent.

30. A pharmaceutical liposome composition, comprising an imexon or a derivative thereof in combination with one or more lipids.

31. A method of treating an individual with cancer, comprising administering to said individual a therapeutically effective amount of a composition comprising an imexon or derivative thereof in combination with one or more lipids.

32. A method of stimulating the immune system of an individual, comprising administering a therapeutically effective amount of a composition comprising an imexon or derivative thereof in combination with one or more lipids.
